(19) United States
(12) Patent Application Publication
(10) Pub. No.: US 2025/0143539 A1
(43) Pub. Date: May 8, 2025

(54) METHODS FOR PLANNING AND GUIDING ENDOSCOPIC PROCEDURES THAT INCORPORATE IMAGE-TO-BODY DIVERGENCE REDUCTION

(71) Applicant: The Penn State Research Foundation, University Park, PA (US)

(72) Inventors: William E. Higgins, State College, PA (US); Yuxuan He, State College, PA (US)

(21) Appl. No.: 18/958,829

(22) Filed: Nov. 25, 2024

Related U.S. Application Data
(63) Continuation of application No. PCT/US24/36761, filed on Jul. 3, 2024.
(60) Provisional application No. 63/512,355, filed on Jul. 7, 2023.

(51) Int. Cl. A61B 1/00; A61B 1/267

(52) U.S. Cl. CPC A61B 1/00006; A61B 1/2676

(57) ABSTRACT

A method for planning and guiding an endoscopic procedure includes obtaining a first scan of a chest cavity at near total lung capacity, obtaining a second scan of the chest cavity at near functional residual capacity, generating a virtual chest cavity based at least in part on the first scan and the second scan, defining one or more regions of interest (ROI) in the virtual chest cavity, determining a route to the one or more ROIs based on the virtual chest cavity, directing endoscope hardware through an airway to the one or more ROIs along the predetermined route, monitoring, by a processor, a real-time position of the endoscope hardware relative to an expected position along the predetermined route, and performing an examination of the one or more ROIs when the real-time position of the endoscope hardware corresponds to an expected ROI location.

### CROSS-REFERENCE TO RELATED APPLICATIONS

This application is a bypass continuation of PCT/US24/36761, filed Jul. 3, 2024, which claims the priority benefit of U.S. Provisional Application No. 63/512,355, entitled METHODS FOR THE PLANNING AND GUIDANCE OF ENDOSCOPIC PROCEDURES THAT INCORPORATE IMAGE-TO-BODY DIVERGENCE REDUCTION, filed on Jul. 7, 2023, the entirety of which is hereby incorporated by reference.

### GOVERNMENT SPONSORSHIP

This invention was made with government support under Grant No. CA151433 awarded by the National Institutes of Health. The Government has certain rights in the invention.

### FIELD OF THE INVENTION

This disclosure relates generally to surgical procedure planning and guiding and, in particular, to a planning and/or guiding methodology that is robust to image-to-body divergence, as applicable to endoscopy.

### BACKGROUND OF THE INVENTION

Many surgical procedures use an endoscope to navigate through hollow tubular structures, such as airways, and/or hollow regions, such as a stomach, situated within the confines of a multi-organ cavity. Such multi-organ cavities are made up of soft tissues and, hence, are subject to dynamic shape changes over time. Various challenges are associated with performing endoscopic procedures in such environments. As such, assisted endoscopy systems are utilized to improve procedure outcomes, and sometimes preferred over standard, unassisted endoscopy procedures. Assisted endoscopy systems draw on computer-based guidance information to facilitate faster and more accurate endoscope navigation to surgical sites of interest. Such systems can develop at least a part of the guidance information from a procedure plan derived off-line prior to a live procedure. The procedure plan can be at least partially derived using a subject's radiologic imaging scan.

However, the real cavity space encountered during the live procedure is typically different than the state reflected in a virtual three dimensional (3D) cavity space. Differences between the real cavity space and the virtual 3D cavity space define the phenomenon referred to as CT-to-body divergence (CTBD), or, more generically, image-to-body divergence. CTBD can be described as the differences between static CT images used for planning and an actual procedure site during a live endoscopy intervention that lead to discrepancies between the actual and expected location of target lesions. Stated another way, CTBD is the difference between a predicted location of a target region of interest during navigation procedure planning (e.g., virtual reality) and an actual target location during endoscopy.

Image-to-body divergence (CTBD) encountered by assisted endoscopy systems limits an ability to localize particular lesions. As such, while performing an endoscopy, the clinician may believe he or she has localized a target based on the assisted endoscopy system's feedback, and, hence, proclaim a navigation success. In reality, however, because of CTBD, the endoscopy misses the target, resulting in a nondiagnostic biopsy.

Endoscopic procedures that take place inside hollow regions located within a multi-organ cavity made up of flexible soft tissues have a performance in confirming, localizing, and/or conducting a biopsy of clinical sites of interest degraded by image-to-body divergence. In particular, substantial errors can arise because of a multi-organ cavity's volume differences observed between an imaging scan, used for planning a procedure, and the actual anatomy, observed during the live procedure. As such, there is a need for a method and/or system that effectively compensates for these differences in assisted endoscopy systems.

### SUMMARY OF THE INVENTION

According to a first aspect of the disclosure, a method for planning and guiding an endoscopic procedure is disclosed. The method includes obtaining a first scan of a chest cavity at a near total lung capacity, obtaining a second scan of the chest cavity at a near functional residual capacity, generating a virtual chest cavity based at least in part on the first scan and the second scan, defining one or more regions of interest (ROI) in the virtual chest cavity, and determining a route to the one or more ROIs based on the virtual chest cavity. The method further includes directing endoscope hardware through an airway to the one or more ROIs along the predetermined route, monitoring, by a processor, a real-time position of the endoscope hardware relative to an expected position along the predetermined route, and performing an examination of the one or more ROIs when the real-time position of the endoscope hardware corresponds to an expected ROI location.

According to a second aspect of the disclosure, a method of planning and guiding an endoscopic procedure is disclosed. The method includes obtaining a first scan of an organ cavity at a first state, obtaining a second scan of the organ cavity at a second state, wherein the first state is different than the second state, generating a virtual organ cavity based at least in part on the first scan and the second scan, defining one or more regions of interest (ROI) in the virtual organ cavity, and determining a route to the one or more ROIs based on the virtual organ cavity. The method further includes directing endoscope hardware along a pathway to the one or more ROIs along the predetermined route, monitoring, by a processor, a real-time position of the endoscope hardware relative to an expected position along the predetermined route, and performing an examination of the one or more ROIs when the real-time position of the endoscope hardware corresponds to an expected ROI location.

According to a third aspect of the disclosure, a method of planning and guiding an endoscopic procedure is disclosed. The method includes obtaining a first scan of an organ cavity at a first state, obtaining a second scan of the organ cavity at a second state, wherein the first state is different than the second state, computing an anatomical model for each of the first scan and the second scan, defining one or more regions of interest (ROI) in the organ cavity, deriving a 3D transformation that maps each point in the first scan to each corresponding point in the second scan, and deriving a procedure plan including a primary route to the one or more ROIs. The method further includes directing endoscope hardware along a pathway to the one or more ROIs, wherein the pathway extends along the primary route, monitoring, by a processor, a real-time position of the endoscope hardware relative to an expected position along the primary route, and performing a desired examination of the one or more ROIs when the real-time position of the endoscope hardware corresponds to an expected ROI location.

According to a fourth aspect of the disclosure, a non-transitory, processor-readable storage medium is disclosed. The non-transitory, processor-readable storage medium comprises one or more programming instructions stored thereon that, when executed, cause a processing device to generate a virtual organ cavity based at least in part on a first scan of an organ cavity at a first state and a second scan of the organ cavity at a second state, wherein the first state is different than the second state, define one or more regions of interest (ROI) in the virtual organ cavity, determine a route to the one or more ROIs based on the virtual organ cavity, and monitor a real-time position of endoscope hardware relative to an expected position along the predetermined route.

According to a fifth aspect of the disclosure, a system for planning and guiding an endoscopic procedure is disclosed. The system includes a processing device and a non-transitory, processor-readable storage medium. The storage medium includes one or more programming instructions stored thereon that, when executed, cause the processing device to generate a virtual organ cavity based at least in part on a first scan of an organ cavity at a first state and a second scan of the organ cavity at a second state, wherein the first state is different than the second state, define one or more regions of interest (ROI) in the virtual organ cavity, determine a route to the one or more ROIs based on the virtual organ cavity, and monitor a real-time position of endoscope hardware relative to an expected position along the predetermined route.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1A is a CT scan of a chest cavity of a subject reflecting a chest volume near TLC according to one or more aspects shown and described herein;

FIG. 1B is a CT scan of the chest cavity of the subject of FIG. 1A reflecting a chest volume near FRC according to one or more aspects shown and described herein;

FIG. 2A is a rendering of the chest volume near TLC captured in FIG. 1A superimposed over a rendering of the chest volume near FRC captured in FIG. 1B according to one or more aspects shown and described herein;

FIG. 2B is an image reflective of the CT scans of FIGS. 1A and 1B superimposed on one another according to one or more aspects shown and described herein;

FIG. 3A is a 3D surface rendering of a segmented airway tree reflecting a chest volume near TLC according to one or more aspects shown and described herein;

FIG. 3B is a 3D surface rendering of a segmented airway tree reflecting a chest volume near FRC according to one or more aspects shown and described herein;

FIG. 3C is a 3D surface rendering of a segmented airway tree after transforming the rendering of FIG. 3A into an FRC space according to one or more aspects shown and described herein;

FIG. 4A is a two dimensional (2D) coronal CT section and segmented airway tree for a subject's TLC CT scan according to one or more aspects shown and described herein;

FIG. 4B is a 2D coronal CT section and segmented airway tree for a subject's FRC CT scan according to one or more aspects shown and described herein;

FIG. 5A is a 3D chest CT scan according to one or more aspects shown and described herein;

FIG. 5B is a rendering of segmented lungs, an airway tree, and an aorta derived from the 3D chest CT scan of FIG. 5A according to one or more aspects shown and described herein;

FIG. 5C is a rendering of segmented bones derived from the 3D chest CT scan of FIG. 5A according to one or more aspects shown and described herein;

FIG. 5D is a rendering of a segmented thoracic cavity derived from the 3D chest CT scan of FIG. 5A according to one or more aspects shown and described herein;

FIG. 6 is a schematic of a tree data structure according to one or more aspects shown and described herein;

FIG. 7A is a still-image captured from a live bronchoscopic video frame I_{V}^{Θ′}^{ⱼ} according to one or more aspects shown and described herein;

FIG. 7B is a VB view I_{CT}^{Θ}^{ⱼ} generated at a virtual space pose Θⱼ for a view site on a predetermined route according to one or more aspects shown and described herein;

FIG. 7C is a 3D CT-based airway tree showing the predetermined route to a particular region of interest (ROI) according to one or more aspects shown and described herein;

FIG. 8 is a schematic showing a chest space and a whole-body space for transformation according to one or more aspects shown and described herein;

FIG. 9A is a segmented airway tree based off of a subject's TLC CT scan according to one or more aspects shown and described herein;

FIG. 9B is a segmented airway tree based off of a subject's FRC CT scan according to one or more aspects shown and described herein;

FIG. 9C is a rigid alignment of the segmented airway trees of FIGS. 9A and 9B after applying a transformation according to one or more aspects shown and described herein;

FIG. 10A is a fully registered volume rendering of a subject's organs for a TLC volume according to one or more aspects shown and described herein;

FIG. 10B is a coronal front-to-back slab view depicting volume differences between a fully registered TLC volume and an FRC volume derived from FIG. 10A according to one or more aspects shown and described herein;

FIG. 11 is an excerpt for a guidance instruction cue set according to one or more aspects shown and described herein;

FIG. 12A is a schematic of a primary guidance route according to one or more aspects shown and described herein;

FIG. 12B is a schematic of a final guidance route according to one or more aspects shown and described herein;

FIG. 13 is a schematic of one or more predetermined back-up routes according to one or more aspects shown and described herein;

FIG. 14A is a 3D surface rendering of an airway tree for depicting on a guidance computer display according to one or more aspects shown and described herein;

FIG. 14B is an integrated view depicting a live bronchoscopic video for depicting on a guidance computer display according to one or more aspects shown and described herein;

FIG. 14C is a 2D axial-plane CT section for depicting on a guidance computer display according to one or more aspects shown and described herein;

FIG. 14D is a coronal slab rendering for depicting on a guidance computer display according to one or more aspects shown and described herein;

FIG. 15 is a schematic representing a volume management tool configured to coordinate synchronized views between TLC-space and FRC-space views according to one or more aspects shown and described herein;

FIG. 16A is a coronal depth-weighted slab view of a chest cavity in TLC space according to one or more aspects shown and described herein;

FIG. 16B is a sagittal slab view of the chest cavity in the TLC space of FIG. 16A according to one or more aspects shown and described herein;

FIG. 16C is a transverse slab view of the chest cavity in the TLC space of FIG. 16A according to one or more aspects shown and described herein;

FIG. 16D is a 3D surface rendered airway tree with a path extending therethrough in the TLC space of FIG. 16A according to one or more aspects shown and described herein;

FIG. 17A is a coronal depth-weighted slab view of a chest cavity in FRC space according to one or more aspects shown and described herein;

FIG. 17B is a sagittal slab view of the chest cavity in the FRC space of FIG. 17A according to one or more aspects shown and described herein;

FIG. 17C is a transverse slab view of the chest cavity in the FRC space of FIG. 17A according to one or more aspects shown and described herein;

FIG. 17D is a 3D surface rendered airway tree depicting a version of the TLC-space path of FIG. 16D mapped into the FRC space of FIG. 17A according to one or more aspects shown and described herein;

FIG. 18A is a 3D airway tree rendering depicting a model of a bronchoscope tip to model if the bronchoscope tip is expected to fit along a desired route according to one or more aspects shown and described herein;

FIG. 18B is a local peripheral renderer depicting a rendering of the airway tree of FIG. 18A, bronchoscope-tip model, and a ROI at a final destination according to one or more aspects shown and described herein;

FIG. 18C is a live bronchoscope video with a guidance cue according to one or more aspects shown and described herein;

FIG. 18D is a panorama renderer depicting an expanded field-of-view VB view at the final destination of FIG. 18B according to one or more aspects shown and described herein;

FIG. 18E is a 2D axial-plane CT section according to one or more aspects shown and described herein;

FIG. 18F is a CT-based simulated rEBUS view at the final destination of FIG. 18B and a live rEBUS view after engaging a probe according to one or more aspects shown and described herein;

FIG. 19A is a 3D rendered airway tree highlighting an active guidance route and current view site, a coronal front-to-back CT slab rendering, and a VB view and guidance route in a TLC space according to one or more aspects shown and described herein;

FIG. 19B is a 3D rendered airway tree highlighting an active guidance route and current view site, a coronal front-to-back CT slab rendering, and a VB view and guidance route in an FRC space according to one or more aspects shown and described herein;

FIG. 20A is a 3D rendered airway tree highlighting an active guidance route and current view site, a coronal front-to-back CT slab rendering, and a VB view and guidance route in a TLC space according to one or more aspects shown and described herein;

FIG. 20B is a 3D rendered airway tree highlighting an active guidance route and current view site, a coronal front-to-back CT slab rendering, and a VB view and guidance route in an FRC space according to one or more aspects shown and described herein;

FIG. 21A is a 3D rendered airway tree highlighting an active guidance route and current view site, a coronal front-to-back CT slab rendering, and a VB view and guidance route in a TLC space according to one or more aspects shown and described herein;

FIG. 21B is a cube view of a 2D coronal, sagittal, and axial plane CT sections at a current view site and a local horizontal 2D CT cross-section centered about the current view site, wherein all views are shown in the TLC space of FIG. 21A according to one or more aspects shown and described herein;

FIG. 21C is a 3D rendered airway tree highlighting an active guidance route and current view site, a coronal front-to-back CT slab rendering, and a VB view and guidance route in an FRC space according to one or more aspects shown and described herein;

FIG. 21D is a cube view of a 2D coronal, sagittal, and axial plane CT sections at a current view site and a local horizontal 2D CT cross-section centered about the current view site, wherein all views are shown in the FRC space of FIG. 21B according to one or more aspects shown and described herein;

FIG. 22A is a 3D rendered airway tree highlighting an active guidance route and current view site, a coronal front-to-back CT slab rendering, and a VB view and guidance route in an FRC space according to one or more aspects shown and described herein;

FIG. 22B is a cube view of a 2D coronal, sagittal, and axial plane CT sections at a current view site and a local horizontal 2D CT cross-section centered about the current view site, wherein all views are shown in the FRC space of FIG. 22A according to one or more aspects shown and described herein;

FIG. 23A is a 3D rendered airway tree highlighting an active guidance route and current view site, a coronal front-to-back CT slab rendering, and a VB view and guidance route in a TLC space according to one or more aspects shown and described herein;

FIG. 23B is a 3D rendered airway tree highlighting an active guidance route and current view site, a coronal front-to-back CT slab rendering, and a VB view and guidance route in an FRC space according to one or more aspects shown and described herein;

FIG. 24 is a flow chart of a method for planning and guiding a bronchoscopic procedure according to one or more aspects shown and described herein; and

FIG. 25 is a flow chart of a method for planning and guiding an endoscopic procedure according to one or more aspects shown and described herein.

### DETAILED DESCRIPTION OF THE INVENTION

The present disclosure, in at least one form, is related to methods and/or systems for planning and guiding an endoscopic procedure. Such methods and/or systems assist a clinician in navigating an endoscope through hollow regions defined inside a multi-organ cavity, where the objective of the endoscopic procedure is to examine one or more predetermined regions of interest (ROI). As the multi-organ cavity may change in shape over time, the described methods and/or systems compensate for image-to-body differences that can arise between how the multi-organ cavity appears in imaging scans used to plan the procedure and how the same multi-organ cavity appears during a live endoscopic procedure.

The various methods disclosed include one or more steps that may be implemented on one or more computing devices. For example, one or more steps of the various methods may be implemented on a plurality of computing devices communicatively coupled to one another. Each of the various computing devices may contain a plurality of hardware components for operation, including, for example, one or more processing devices, at least one non-transitory memory component, network interface hardware, device interface hardware, and/or at least one data storage component. In some embodiments, each of the various computing devices may include hardware that provides a user interface such that information can be provided to one or more users and/or such that inputs can be received from one or more users. A local interface, such as a bus or the like, may interconnect the various components.

In some embodiments, the various processes may be contained within one or more non-transitory, processor-readable storage mediums as program code.

The present disclosure has many advantages and unique attributes relative to other assisted endoscopy systems. Notably, the described methods and/or systems rely on computer-based software methods, without the need for extra hardware to make body measurements or for sensors attached to the endoscopic devices or to the human body, as needed by ENB systems. Hence, the described methods and/or systems are not subject to errors in the sensor/body location measurements made by these devices during a live procedure and require less hardware. Additionally, the described methods and/or systems make more exhaustive use of the known subject-specific airway tree structure and branch hierarchy, thereby enabling extra location certainty during a procedure. Related to this point, the described methods and/or systems also make better use of the available knowledge of the local airway tree's branch structure. The described methods and/or systems do not rely on the global overall shape and absolute size of the thoracic cavity and its constituent organs. Instead, the described methods and/or systems rely on the anatomy's fundamental top-level invariant 3D topology, which does not change as a subject inhales and exhales. Pre-operative procedure planning enables the derivation of alternate back-up routes prior to the live procedure to account for possible blocked, narrowed airways, and other unexpected events during the live procedure. During live guidance, the described guidance system automatically suggests alternate guidance routes, thereby facilitating on-the-fly route changes during the live procedure. The described methods and/or systems can work in tandem with existing robotics assisted bronchoscopy systems, thereby enabling automatic navigation assistance. The described methods and/or systems provide a high-quality mechanism, geared toward the subject's live FRC-based lung state for final target confirmation/localization, as the system's guidance information seamlessly switches from a TLC-based thoracic model to a more realistic FRC-based model when the target ROI becomes close. More detailed local graphical information is also provided to assist with final ROI examination and/or rEBUS analysis. The described methods and/or systems simultaneously provide augmented reality-based graphical information on procedure progress for both the TLC and FRC spaces, where the current guidance state is synchronized to both spaces. Furthermore, the described methods and/or systems enable rapid guidance and deployment of rEBUS probe placement to enable live confirmation of extraluminal targets.

In various instances, the hollow regions can be lung airways, a colon, a stomach, vasculature in the heart, lungs, a brain, a liver, a kidney, or any other hollow areas situated inside a multi-organ cavity. The multi-organ cavity can be a thoracic cavity, including the mediastinum, an abdominal cavity, a pelvic cavity, or a cranial cavity, for example. Exemplary ROIs include a suspect tumor, a cancer nodule, a suspicious airway wall site, or an anatomic location of interest for general visual examination, tissue biopsy, and/or treatment delivery. Examination of the ROI by the endoscope may include collecting anatomical tissue from the ROI, performing a visual assessment of the ROI, and/or delivering treatment to the ROI.

According to aspects of the present disclosure, coordinated planning and guidance methodologies, incorporated into a unique assisted endoscopy system that facilitates pre-operative procedure planning and subsequent live intra-operative guidance of endoscopy for the purpose of examining a target region of interest (ROI), are described.

The disclosure focuses on assisted bronchoscopy systems, as they apply to applications in the chest, or thoracic cavity, and to lung cancer management including diagnosis, staging, monitoring, follow up, and/or treatment, for example. In particular, the present disclosure describes a unique approach for overcoming anatomical volume differences observed in a subject's imaging scan (a virtual space) as compared to the anatomy's appearance and/or shape during a live bronchoscopy procedure (a real space). However, it is envisioned that the disclosed systems and methods can be used for any surgical procedure involving a surgical instrument, such as an endoscope. In various instances, the endoscope can be a bronchoscope, colonoscope, laparoscope, angioscope, catheter, or cystoscope, for example.

The disclosed systems and methods are envisioned for use during surgical procedures such as a bronchoscopy performed in an airway tree, which is within a thoracic cavity, a laparoscopy performed in the stomach, which is within an abdominal cavity, a colonoscopy performed in a colon, which is within the abdominal cavity, a cystoscopy performed in a bladder, which is within a pelvic cavity, and/or a cardiac catheterization performed in a heart, which is within the thoracic cavity, for example.

Lung cancer remains the world's largest cause of cancer death, being responsible for over 30% of all cancer deaths. Notably, lung cancer currently has a poor 5-year survival rate on the order of 15%. Hence, early detection of lung nodules, such as tumors and/or lesions, has been recognized by the medical community as being vital for diagnosing curable lung cancer. In 2011, the National Lung Screening Trial (NLST) showed that screening via low-dose computed tomography (CT) scans significantly increases early detection and reduces mortality from malignant tumors. This effort has spurred a major paradigm shift in early lung cancer detection, manifested by an ongoing global roll-out of CT-based lung cancer screening programs.

With this shift, tens of millions of subjects at high risk for lung cancer now have the opportunity for early disease detection and, in turn, a greater likelihood of survival. However, tumor detection via image-based CT screening only raises the suspicion that a subject may have lung cancer. Therefore, further follow-up clinical steps involving tissue diagnosis are required. These steps can include, for example: 1) peripheral nodule diagnosis to confirm the disease; and 2) central chest lymph node staging, to determine disease extent and treatability. In addition, some subjects may require additional follow-up disease monitoring/follow-up and cancer treatment.

Notably, the NLST reported that nearly 40% of CT-screened subjects had at least one detected lesion, with nearly 80% of these lesions being peripheral nodules. Such subjects often require tissue biopsy to confirm their disease state. Percutaneous nodule biopsy is the long-standing de facto approach for sampling peripheral nodules. However, such approach is associated with an unfavorable profile. As such, clinicians now often prefer to use minimally invasive bronchoscopy for conducting a biopsy of many peripheral lung nodules. The recent introduction of lung cancer screening is greatly increasing the number of subjects presenting a suspect nodule, which in turn is greatly increasing the volume of bronchoscopies required to manage these subjects.

It is known, however, that a high percentage of observed nodules do not evolve into malignant cancer. Hence, it is vital that bronchoscopy-based peripheral nodule biopsy be accurate. Unfortunately, an unacceptable number of bronchoscopies, without any other assisting guidance aides, are known to be unsuccessful, with yields below 50% commonly reported. Such a shortcoming delays early disease detection and can seriously affect a subject's long-term prognosis.

In an effort to address this shortcoming, assisted bronchoscopy systems have been developed, drawing on CT-based virtual bronchoscopy (VB). Previously, image-guided bronchoscopy systems were introduced, with FDA-approved systems now readily available. More recently, new robotics-assisted bronchoscopy systems also began to become feasible, especially for examining distant peripheral sites and for performing more complex chest procedures.

Assisted endoscopy systems can be classified into two forms. Image-guided endoscopy systems involve a user, such as a clinician, navigating the endoscope. Robotics-assisted endoscopy systems involve a machine, or robot, navigating the endoscope under user control. Stated another way, the robot navigates the endoscope under user supervision and/or in response to a user input, for example.

As described herein, for any type of assisted endoscopy system, two phases of operation occur: (1) off-line procedure planning; and (2) live guided endoscopy. During phase 1 off-line procedure planning, a clinician first creates a procedure plan off-line prior to the live endoscopy. To create the procedure plan, the clinician first selects one or more diagnostic regions of interest (ROIs), such as a suspect nodule and/or any other anatomical site of interest located inside a cavity, such as a thoracic cavity. The one or more diagnostic ROIs are selected using the subject's three-dimensional (3D) chest computed tomography (CT) scan and/or the subject's positron emission tomography (PET) scan, if available, such as in a co-registered PET/CT study.

Next, for each of the one or more ROIs, the subject's CT scan is used to create an airway route leading through the lung airways to the particular ROI. Such an airway route can be derived automatically via computer analysis and/or manually via visual inspection. The created airway route provides a virtual pathway through the lung airways leading to the particular ROI in a 3D CT-based virtual chest space. Assisted bronchoscopy systems in general have a computer-based software system that helps in the derivation of the procedure plan.

During phase 2 live guided bronchoscopy, the assisted bronchoscopy system is actuated, or otherwise engaged, in a procedure room, such as an operating room. As discussed in greater detail herein, the assisted bronchoscopy system can be an image-guided system and/or a robotics-assisted system. The assisted bronchoscopy system can be interfaced to bronchoscopy hardware to be able to tap off live video sources, for example.

Based at least in part by a display and/or the assisted bronchoscopy system's procedure plan, the clinician navigates a bronchoscope along the predetermined, or preplanned, route, such as along the suggested airways, leading to the targeted, particular ROI. Fundamentally, this entails synchronizing the bronchoscope's actual position within the subject, which is in a real, physical chest space, to the information driving the procedure plan, which is in a CT-based virtual chest space.

Upon navigating close to, and/or reaching, the particular ROI near the terminus of the preplanned route, a precise position of the site of the particular ROI is confirmed, or localized. As most nodules tend to be external to the airway—and, hence, not visible in the endoluminal field of view of the bronchoscope—the clinician relies on a second complementary imaging device that gives live 2D extraluminal views. The second complementary imaging device provides information regarding an opposing, external side of the airway lumen to make a final confirmation of the target lesion, for example. Exemplary secondary imaging devices include radial probe endobronchial ultrasound (EBUS), often referred to as rEBUS or RP-EBUS, CT fluoroscopy, and cone beam CT (CBCT).

After confirming the precise position, or location, of the particular ROI, the clinician can perform a desired examination, such as a tissue biopsy, for example.

As described, considerable progress has been made in the development of assisted bronchoscopy systems and the supplemental confirming imaging devices. As such, it is well-recognized that guiding bronchoscope navigation to a particular ROI to within approximately 2-4 cm of the final target location is essentially a solved problem. Unfortunately, however, final target ROI confirmation and/or localization, performed over the final 2-4 cm along an ROI's airway route, still poses considerable difficulty for assisted bronchoscopy systems. As an example, the multi-center NAVIGATE study found a 12-month diagnostic yield of 73% using assisted bronchoscopy systems, implying a nearly 30% failure in properly localizing desired biopsy sites. Pertaining to assisted bronchoscopy systems, it has been acknowledged that in the past two decades, multiple bronchoscopy techniques focused on the diagnosis of peripheral lung nodules have been developed. Although peripheral bronchoscopy has been established as an effective procedure, its diagnostic yield seems to have plateaued at about 70%.

One factor that affects final target ROI confirmation and/or localization is that all of the aforementioned supplemental imaging devices have deficiencies.

More specifically, while rEBUS provides views within a risk of exposure to radiation, rEBUS can provide false-positive confirmation images of ghost “nodules,” arising from a common phenomenon known as atelectasis. Atelectasis is the partial regional collapse of a lung caused by the closing of bronchioles or pressure outside of the lung. Atelectasis (and the production of false rEBUS views) was observed in over 70% of considered subjects, with atelectasis becoming more likely for subjects having a higher BMI (body mass index) and/or under general anesthesia for a longer period of time (>30 min).

CT fluoroscopy uses X-ray radiation for garnering confirmation images. Unfortunately, this subjects the subject and clinicians to extra radiation. Further, CT fluoroscopy only gives 2D projection views of the target ROI region. Such 2D projection views are well-known to be potentially misleading.

While a portable CBCT scanner, which entails performing a small local CT scan near the target site, can give helpful confirmation views, the portable CBCT scanner also entails exposure to considerable radiation and/or a significant hardware expense.

Another disadvantage to using the above-discussed secondary imaging devices is that such imaging devices are not directly linked to the assisted bronchoscopy's system guidance or display protocol in any way. As such, an onus is on the clinician to deploy a particular device accurately about the target ROI confirmation and/or localization site.

An additional factor contributing to the relating to the low success rates associated with the use of current assisted bronchoscopy systems relates to the changing dynamic nature of the thoracic cavity. Such a dynamic nature perhaps has the greatest impact on final target ROI confirmation and/or localization. More specifically, all assisted bronchoscopy systems depend on the fundamental principle of synchronizing, or registering, the preplanned airway route information derived with respect to the virtual chest space to the bronchoscope's real-time position in the subject's chest space.

As discussed herein, a subject's 3D chest CT scan and optional PET/CT study delineate the virtual 3D chest space. A subject's real, or actual, chest space encountered during the live procedure can be defined by one or more bronchoscope/EBUS video streams and/or acquired live body shape/position measurements as done by so-called electromagnetic navigation bronchoscopy (ENB) systems. Such live body shape and/or position measurements can be derived by airway landmark/fiducial locations and/or sensors mounted on a chest or attached to an instrument.

To delineate the virtual 3D chest space, it is standard radiology protocol for the chest CT scan to be generated by having a subject perform a short breath hold. As such, the imaged lungs are near full inspiration, or near total lung capacity (TLC)—a state where the lungs are near their largest total volume.

However, the real chest space encountered during the live procedure is typically in a different state than the state reflected in the virtual 3D chest space. Notably, during the live procedure the subject breathes freely with his or her lungs staying at a relatively low volume, also referred to as tidal breathing. In other words, the lung volume of the subject generally stays near a functional residual capacity (FRC), or near end expiration, reflective of the smallest total lung volume at all times during the procedure. As such, all live procedural data relating to the subject's chest, which defines the real chest space, is derived from a state near FRC.

Such differences between the information used to derive the virtual and real chest spaces highlight a fundamental issue giving rise to the performance limitation highlighted. For peripheral nodule biopsy, this limitation is also present in the “TLC-mapped” ENB systems for assisted bronchoscopy. More specifically, the movement of lesions has been observed in a subject's joint pair of inspiratory (near TLC) and expiratory (near FRC) CT scans. Such a lesion's position differed on average by 17.6 mm between the scans, with the average difference≥23.6 mm for the left/right lower lung lobes lesions. Considering that typical nodules tend to be on the order of 10 mm in diameter, this large error indicates that many nodules could easily be missed during bronchoscopy, depending on how the imaging scan used for procedure planning relates to the chest volume encountered during the live procedure.

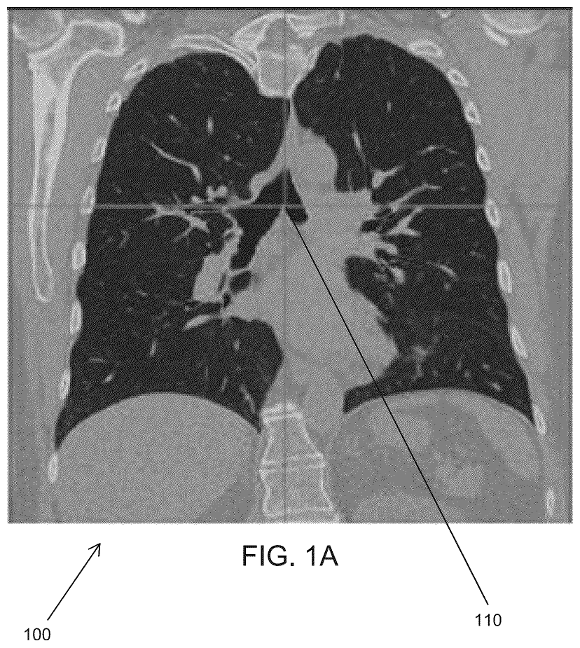

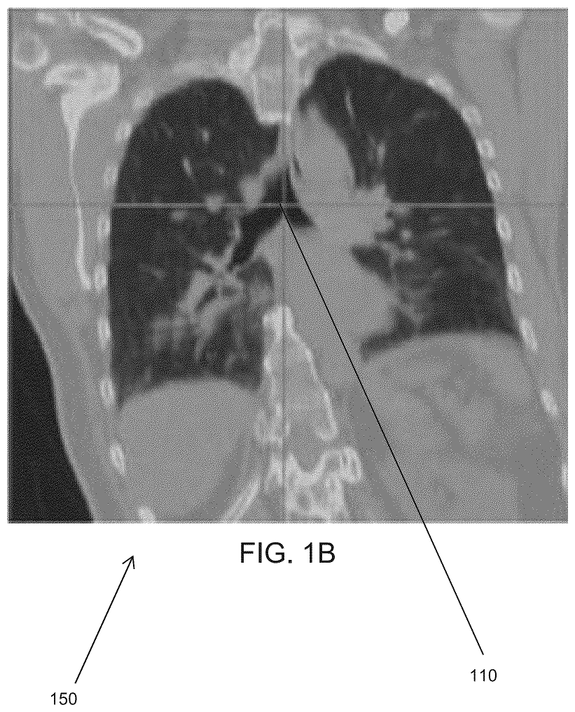

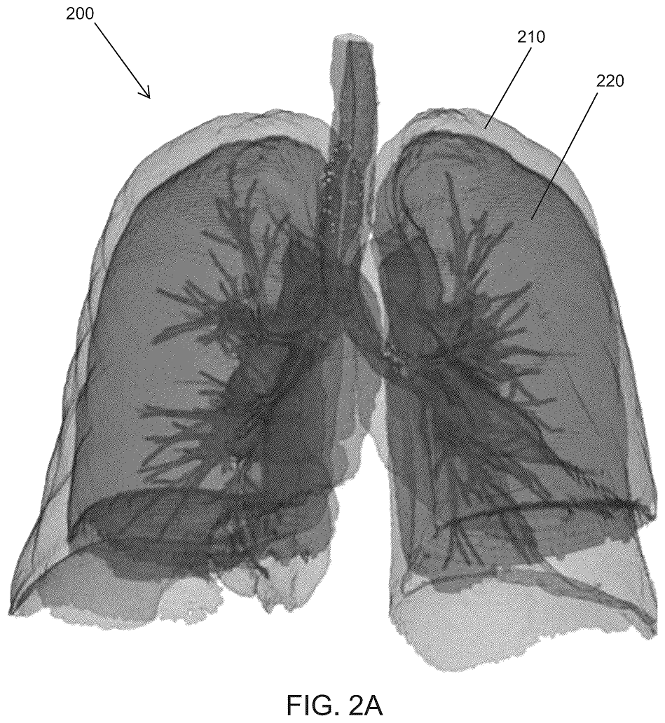

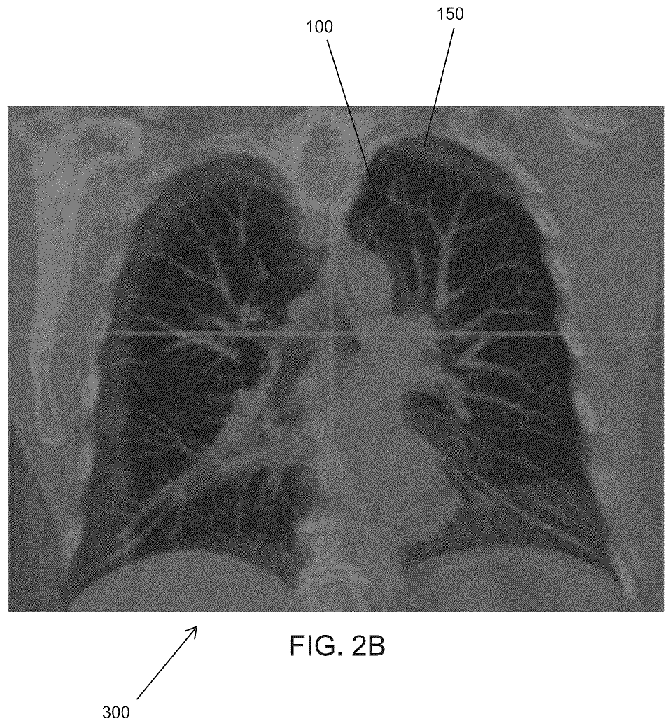

FIGS. 1A and 1B depict CT scan differences of imaged chest volumes between TLC and FRC. More specifically, FIG. 1A is a 2D coronal CT section scan 100 of a TLC chest cavity while FIG. 1B is a 2D coronal CT section scan 150 of a FRC chest cavity. Both scans 100, 150 shown in FIGS. 1A and 1B reflect sections aligned at a same landmark near a main carina, as noted by a cross-hair 110 extending through the scans 100, 150. A difference in the imaged chest volumes represents how the total lung volumes perceived by the virtual (near TLC) and real (near FRC) chest spaces can differ. FIGS. 2A and 2B further demonstrate that this difference is typically substantial, or large—being a 52% lung volume difference from TLC to FRC for this depicted example—with the largest difference noted in a lower lung periphery. FIG. 2A depicts volume renderings 200 of the imaged organs at a TLC state 210 and an FRC state 220, whereas FIG. 2B depicts an image 300 of the scans 100, 150 of FIGS. 1A and 1B superimposed on one another. These differences define the phenomenon referred to as CT-to-body divergence (CTBD), or, more generically, image-to-body divergence. CTBD can be described as the differences between the static CT images used for planning and the dynamic airways during intervention live bronchoscopy that lead to discrepancies between the actual and expected location of target lesions. CTBD can also be described as the difference between the predicted location of the peripheral target during navigation procedure planning (e.g., virtual reality) and the actual target location during bronchoscopy.

It is well-recognized that the image-to-body divergence (CTBD) encountered by assisted bronchoscopy systems is, in some sense, a “fatal flaw” that limits an ability to localize peripheral lung lesions. In particular, while performing a bronchoscopy, the clinician may believe he or she has localized a target based on the assisted bronchoscopy system's feedback, and, hence, proclaim a “navigation success”. In reality, because of CTBD, the bronchoscopy misses the target, resulting in a nondiagnostic biopsy.

The methods and systems described herein present a solution that accommodates, or otherwise compensates, for image-to-body divergence. Stated differently, the methodologies described herein account for the dynamic shape-changing behavior of the thoracic cavity and its constituent organs, including the lungs and/or major airways, to enable robust and accurate bronchoscopy in the face of image-to-body divergence. As a result, the methodologies and systems described herein help to reduce observed bronchoscope position errors (on the order of approximately 20 mm) commonly encountered while trying to localize and/or position the bronchoscope and supplemental confirming imaging devices (e.g., rEBUS) to localize, examine, biopsy, and/or treat a region of interest.

To begin, a method of planning and guiding an endoscopic procedure assumes that the subject has two distinct 3D CT scans available, each depicting a thoracic cavity of the subject at different states. One 3D CT scan showing the thoracic cavity at near TLC and one 3D CT scan showing the thoracic cavity at near FRC. In various instances, the method includes capturing the two distinct scans.

The two CT scans depicting the thoracic cavity are acquired pre-operatively. I_{TLC} is used herein to refer to the CT scan showing the thoracic cavity near TLC, or near full inspiration. I_{FRC} is used herein to refer to the CT scan showing the thoracic cavity near FRC, or near full expiration.

The CT scan I_{TLC} focuses on the chest and thoracic cavity, is generated via the standard radiologic protocol, whereby the subject performs a short breath-hold during the scan, and has high spatial resolution, with both the axial-plane resolutions (Δx, Δy) and 2D section spacing Δz being <1 mm. I_{TLC}, which exhibits the airways at their fullest volume during the breathing cycle, captures the truest attainable representation of the physical airway tree's intrinsic topology. As such, I_{TLC} serves as the reference scan for CT-based virtual space throughout, including during live guidance. In particular, I_{TLC} is the least likely volume to exhibit deviations, such as blocked airways, from the physical airway tree's true intrinsic topology.

CT scan I_{FRC} is also generated pre-operatively. In various instances, the CT scan I_{FRC} is generated in the same scan session that I_{TLC} is generated, as is done for instance for the SpinDrive assisted bronchoscopy system. As such, I_{FRC} also can be a 3D high-resolution volume of the chest, similar to I_{TLC}. In various instances, the CT scan I_{FRC} is obtained by the subject undergoing a separate whole-body PET/CT study, as often prescribed for lung cancer subjects. In such instances, both the PET and CT scans are co-registered, span the whole body (typically knees to nose), and both the PET and CT scans are collected as the subject breathes freely, i.e., tidal breathing. The resulting I_{FRC} typically has adequate axial-plane resolution (Δx, Δy)≈1 mm or less, but the slice thickness Δz is generally >1 mm. Thus, the spatial resolution is generally lower than I_{TLC}. Furthermore, additional blurring artifact arises, because of the free-breathing scanning protocol usually employed for PET/CT studies.

Research has shown that the elasticity of the lungs during the breathing cycle from inspiration to expiration causes regional expansion and contraction to the shapes of the lungs without changing the underlying anatomical structure of the lungs or the airway tree. Thus, a 3D spatially displacement field can readily be derived to relate the constituent elements of TLC and FRC chest CT scans, as discussed herein.

This has led to recent research, whereby a supervised deep-learning-based generative adversarial network (GAN) and a biomechanical model capturing the deformation vector field representing the lung tissue elasticity model could be used to generate acceptable end-inspiration (FRC) CT images very rapidly. A related work also describes a deep learning approach to generate 3D cone beam CT lung images from single 2D projection views. Such approaches could conceivably obviate the need for a precise FRC-based CT scan, as the reference TLC scan is available to give an accurate topological model. As described further herein, navigation is linked between the FRC space and the gold-standard TLC space. Hence, the FRC-based image need not be “perfect.”

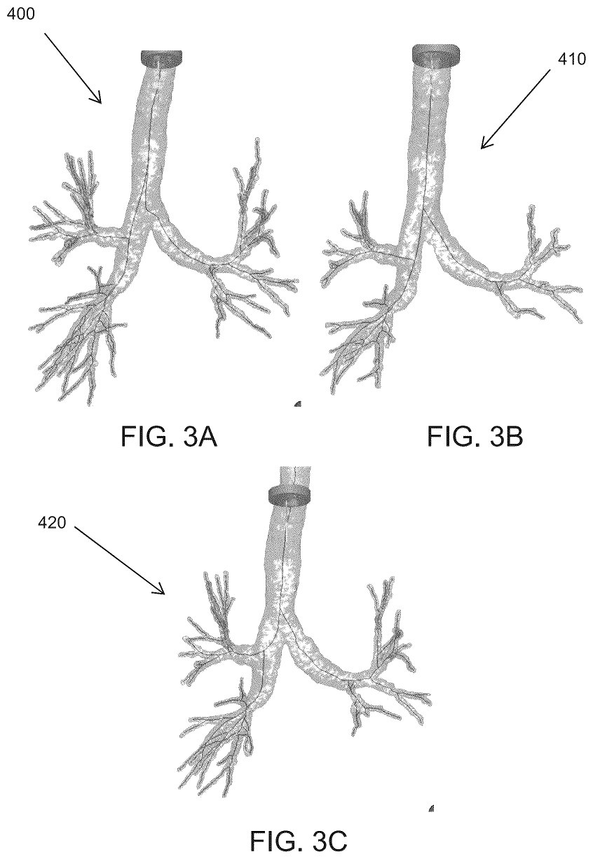

As stated earlier, I_{FRC} depicts the airway tree in a state that is closer in shape/volume to the subject's physical airway tree during live bronchoscopy. This point might suggest that the TLC scan is unnecessary for planning and guiding the live procedure. However, several reasons work against this notion. For instance, a major consequence of imaging the airway tree during the low-volume FRC state is that small-diameter peripheral airways often appear narrowed, compressed, or blocked in I_{FRC}. Thus, certain airways, which appear in I_{TLC}, will appear narrowed or blocked in I_{FRC}. This can in turn result in successor downstream higher-generation airways, which appear in I_{TLC}, to not appear at all in I_{FRC}. These missing airways, however, might still be needed for planning a guidance route for live bronchoscopic navigation toward the ROI. Thus, the FRC scan I_{FRC} often captures an airway tree that has a reduced hierarchy of airway branches; i.e., airways and airway subsegments can appear pruned from the full tree. The airway trees depicted in FIGS. 3A-3C illustrate this phenomenon. The airway trees depicted in FIGS. 3A-3C are the result of CT scans collected at different lung volumes. FIGS. 3A-3C each illustrate a 3D surface rendering of a segmented airway tree and extracted airway centerlines. More specifically, FIG. 3A depicts a result 400 of a subject's TLC CT scan I_{TLC}. The segmented airway tree includes 129 branches b and 65 paths p. FIG. 3B depicts a result 410 of the subject's FRC CT scan I_{FRC}. The segmented airway tree includes 75 branches b and 38 paths p. FIG. 3C depicts a result 420 after transforming the subject's TLC CT scan I_{TLC} into the FRC space. The segmented airway includes 95 branches b and 48 paths p. Both scans I_{TLC} and I_{FRC} are high-resolution breath-hold 2D CT scans, with axial-plane resolution Δx=Δy=0.566 mm and splice spacing Δz=0.60 mm. The TLC and FRC scans include 475 and 448 2D axial-plane sections, respectively, with each 2D section including 512×512 voxels.

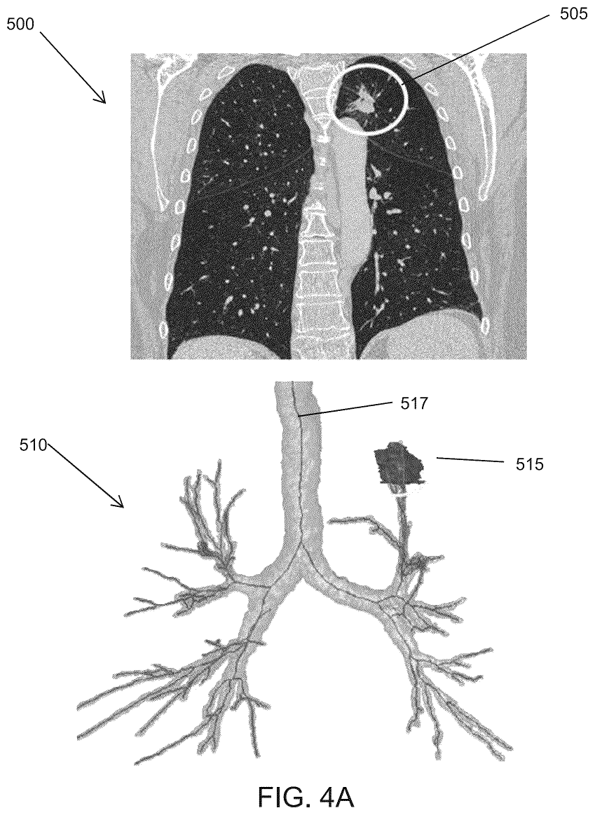

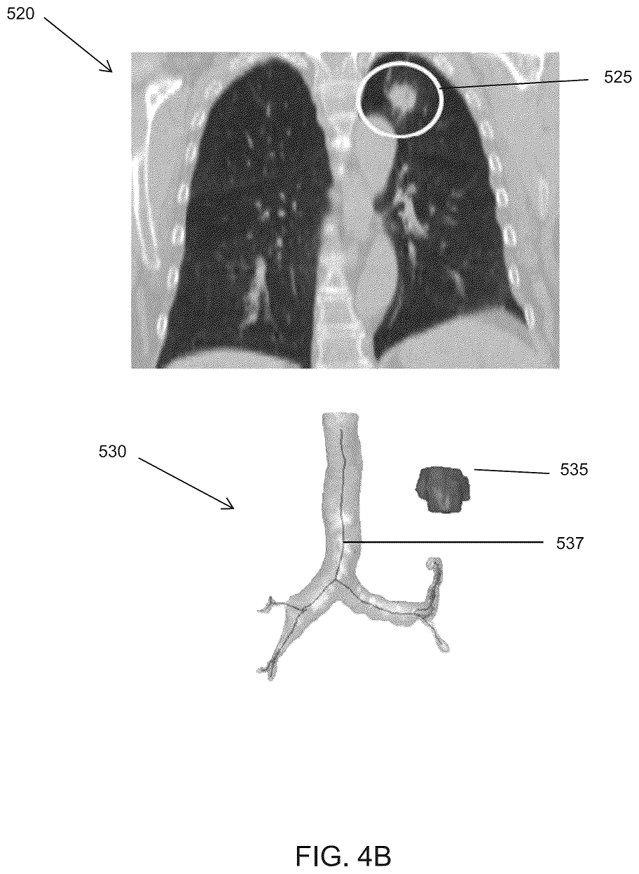

Moreover, if I_{FRC} is derived from a PET/CT study, the given scan is likely to have lower resolution and added motion blur artifact—factors that degrade the quality of the imaged airway tree and airway endoluminal surfaces. This may preclude the possibility of finding a feasible airway pathway (guidance route) leading to a peripheral ROI. FIGS. 4A and 4B illustrate these factors for an example CT scan derived from a PET/CT study. More specifically, FIGS. 4A and 4B illustrate differences between I_{TLC} and I_{FRC} brought about by changes in lung volume, image resolution, and/or breathing motion of a subject. I_{FRC} is derived from a whole-body PET/CT study. FIG. 4A depicts a 2D coronal CT section 500 and a segmented airway tree 510 for the subject's TLC CT scan. FIG. 4B depicts a 2D coronal CT section 520 and a segmented airway tree 530 for the subject's FRC CT scan. For both FIGS. 4A and 4B, circled regions 505, 525 denote a suspect left upper lung (LUL) tumor in the 2D coronal CT sections 500, 520, respectively. Circled regions 515, 535 denote the suspect LUL tumor in the rendered airway trees 510, 530, respectively. Extracted airway centerlines 517, 537 are further highlighted in the rendered airway trees 510, 530, respectively.

In instances where a deep learning approach is used to produce I_{FRC}, it will require a high-resolution input, such as I_{TLC}. In addition, the generated image could have internal artifacts as it is not strictly speaking “the real thing.” Lastly, the generated FRC CT scan will have the same issues as those depicted in and described with respect to FIGS. 3A-3C.

Hence, I_{TLC} is required and serves throughout as the “best” case reference-standard 3D scan. In particular, I_{TLC} gives the most complete delineation of 3D CT-based virtual space in that it depicts the fullest available airway tree. In addition, I_{TLC} serves as the best case basis for the planning and guidance processes.

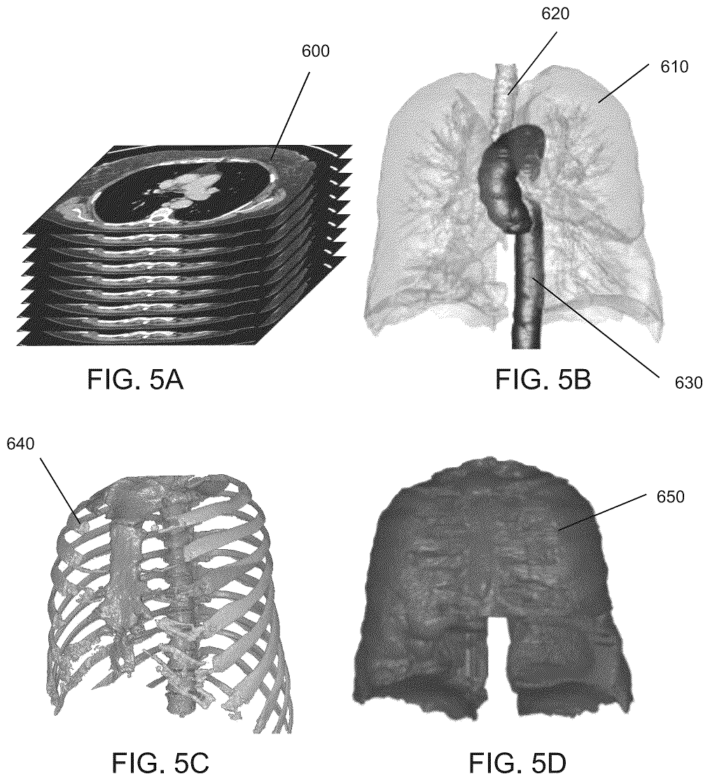

Upon acquiring I_{TLC} and I_{FRC}, the method continues by developing a plan for a particular procedure. As shown in FIGS. 5A-5D, to begin, for each scan 600, existing methods are used to compute a chest model, which includes a 3D segmented airway tree 620, airway endoluminal surfaces, and airway-tree centerlines. In addition, segmentations of the thoracic cavity 650, lungs 610, aorta 630, pulmonary artery, bones 640, and other pulmonary vasculature are computed. These structures along with the associated CT scan define a 3D CT-based virtual space. Next, the clinician defines the desired ROI R, be it a peripheral nodule or any other relevant clinical site of interest, using existing semi-automatic techniques such as the interactive live wire, region growing, and others.

The airway tree 620 serves as a natural navigation space for image-guided bronchoscopy, with airway centerlines acting as the 3D virtual representation of the physical “road network” for the navigation space. Therefore, the airway centerlines, act as a major element in the described methodology. As such, the mathematical underpinnings of this structure are discussed.

The airway tree's hierarchical branching structure can be represented by the data structure given by T=(V, B, P), where for integers vmax, bmax, and pmax≥1. V={v₁, . . . , vᵥₘₐₓ} represents a set of view sites, B={b₁, . . . , b_{bmax}} represents a set of branches, with b₁=generation-1 root branch of tree T, and P={p₁, . . . , pₚₘₐₓ} represents a set of all possible airway paths starting at branch b₁ and ending at a terminal branch.

A view site v=(x, d) specifies a single discrete point within the airway tree, where x signifies the 3D location/coordinates (x, y, z) of v within the 3D volume delineated by the CT scan it is defined within, be it I_{TLC} or I_{FRC}, while d specifies the view direction and orientation. The quantity d includes a vector specifying the view direction and an up vector that denotes an observer's orientation when viewing the scene from view site v. During the process of computing the centerlines, neighboring view sites are discretely spaced nearly an equidistant amount from each other, where their spacing Δv is a function of the CT scan's 3D resolution (Δx, Δy, Δz). In addition, while deriving the centerlines, the airway branch diameter and branch angle for each constituent view site is also computed; hence, this information is also associated with each view site. The view site v can also be referred to as a pose in a virtual space.

A branch

$$b = \{ {v}_{a} , {v}_{b} , … , {v}_{l} \} \qquad ( 1 )$$

is made up of an ordered set of neighboring connected view sites vₐ, v_{b}, . . . , vₗ∈V. The first view site vₐ of root, or parent, branch b₁ is the root site v₁, which starts the entire tree, while b₁'s final view site vₗ is a bifurcation point, which spawns, or otherwise begins, successor child branches constituting the tree. For the lungs, the two child branches of b₁ are the right main bronchus (RMB) and the left main bronchus (LMB). For all other branches b≠b₁, vₐ is a bifurcation point, thereby implying that b has a parent branch, while vₗ can be either a bifurcation point or an end point. Branch b is a terminal branch if vₗ is an end point, implying that b has no child branches. If branch b is not a terminal branch, then vₗ is a bifurcation point and b spawns child branches.

Lastly, a path

$$p = \{ {b}_{1} , … , {b}_{m} \} \qquad ( 2 )$$

includes an ordered hierarchal set of branches b₁, . . . , bₘ∈B. All paths start with root branch b₁, continue with a series of child/ancestor branches, and end with a terminal branch bₘ. For the lungs, a path represents an airway pathway starting in the trachea (root branch b₁) and ending at some terminal airway branch.

For the described methodology, it is necessary to better specify branch equation (1) for a branch bⱼ as the following ordered set of ωⱼ view sites:

$${b}_{j} = \{ {v}_{j , 1} , {v}_{j , 2} , … , {v}_{j , {ω}_{j}} \} \qquad ( 3 )$$

where vⱼ, i∈V is the i^{th} view site constituting bⱼ. With definition (3), the branch length lⱼ of bⱼ can now be defined as the accumulated Euclidean distance

$$Δ v = \vert {v}_{j , i} - {v}_{j , i + 1} \vert \qquad ( 4 )$$

between each connected pair of view sites (v_{j,i}, v_{j,i+1}), i=1, . . . , ωⱼ−1.

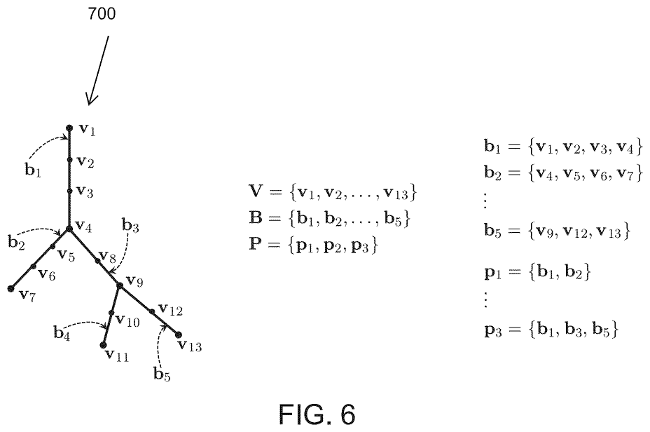

FIG. 6 provides an example of the many tree data structure concepts discussed above. For example, a tree data structure 700 is depicted that is defined by T={V, B, P}, where the set of view sites V includes thirteen (13) view sites, the set of branches B includes five (5) branches, and the set of paths P includes three (3) complete paths through the tree. View sites v₄ and v₉ are bifurcation, or branch, points, while v₄, v₇, v₉, v₁₁, and v₁₃ are endpoints.

Given this development, the related concepts of the virtual/real spaces and the airway guidance route are now introduced. As is standard for assisted bronchoscopy systems, a subject's high-resolution 3D CT scan delineates the virtual (chest) space, while the subject's actual chest anatomy defines the real (chest) space. As an ancillary concept, the airway guidance route is defined as a continuous connected pathway through the airway tree interior that leads to ROI R.

As used herein, r is used to denote an airway guidance route delineating a connected pathway through the airway tree leading to ROI R. Route r consists of an ordered hierarchical set of branches leading from the trachea (branch b₁) to a final destination near the ROI:

$$\begin{matrix}r & = & \{ {b}_{1} , {b}_{2} , … , {b}_{j} , … , {b}_{a} , s \} & ( 5 ) \\ & = & \{ {v}_{1} , … , {v}_{1 , {ω}_{1}} , {v}_{2 , 2} , … , {v}_{2 , {ω}_{2}} , {v}_{3 , 2} , … , {v}_{3 , {ω}_{3}} , … , {v}_{a , {ω}_{a}} , {v}_{a + 1 , 2} , … , {v}_{D} \} & ( 6 ) \\ & & & \end{matrix}$$

where each branch bⱼ∈B, j=1, 2, . . . , a, is made up of view sites per (3) and

$$s = \{ {v}_{a , {ω}_{a}} , {v}_{a + 1 , 2} , … , {v}_{a + 1 , v} , {v}_{a + 1 , v + 1} , … , {v}_{D} \} \qquad ( 7 )$$

is a terminating segment concluding with destination view site v_{D} located close to the ROI.

In various instances, a trivial airway guidance route of the form r={s} made up only of a segment s is possible. Such a route would be appropriate, for example, for a station 2 lymph node, but it would not occur for a peripheral nodule.

As with the paths (2), all routes begin with the airway tree's tracheal branch b₁.

Per (3), the final view site of branch bⱼ, namely vⱼ, ωⱼ, also equals the first view site v_{j+1,1} of bⱼ₊₁. Thus, v₁, ω₁=v_{2, 1}, v₂, ω₂=v_{3, 1}, etc., in (6).

Route r by convention consists of a complete branches bⱼ∈B and a partial (a+1)^{th} “branch” specified by (7).

(6) highlights route r's a endpoints

$${v}_{j , {ω}_{j}} ≡ {v}_{j + 1 , 1} , j = 1 , 2 , … , a \qquad ( 8 )$$

which prove to be key points in constructing alternate (back-up) guidance routes toward the ROI, as discussed in greater detail herein.

A major point to realize is that many possible airway guidance routes r traversing different airway branches could conceivably be feasible, depending on the criteria used to define a destination view site v_{D}.

The terminating segment s is a vital element of the final localization process for optimal ROI examination. As the route computation process elaborates on more below, the first several view sites constituting terminating segment s of (7) will likely be in set V; e.g., the first v view sites of s up to view site v_{a+1,v} in (7) may be view sites constituting the initial portion of some branch b∈B. The concluding series of view sites—e.g., view sites v_{a+1,v+1} through v_{D} in (7)—however, are computed so that they enable the bronchoscope and possibly a biopsy needle or ancillary device such as an rEBUS probe to fit within the local confines of the airway tree and also enable optimal ROI examination. Hence, these view sites are unlikely to be in set V.

Navigation through the airways of the CT-based virtual space is accomplished by a virtual bronchoscope (VB), or virtual camera, where the virtual bronchoscope produces CT-based VB views I_{CT}^{Θ} (“virtual video” views) at each pose Θ along the guidance route. When following a preplanned airway guidance route r of the form (6), the pose Θ is defined by a constituent view site v∈r. As is standard in the field of computer vision, a pose Θ is given by a six-parameter vector defining the pose's 3D spatial (x, y, z) position and Euler angles (α, β, γ). When Θ is defined by a view site v=(x, d), Θ's specifications are given by (x, d).

Analogously, during live bronchoscopy, the physician attempts to navigate through the subject's airways (real space) toward the ROI by following the guidance offered by the assisted bronchoscopy system. In particular, the physician navigates the bronchoscope through the real airway tree by observing real bronchoscopic video camera images I_{V}^{Θ} of the subject's airway tree endoluminal, or interior, structure at various poses Θ imaged by the bronchoscope as it is maneuvered through the subject's airways. That is, I_{V}^{Θ} is a 2D video frame in the bronchoscope's live video stream at pose Θ in real space.

Given the definitions above, the goal of an assisted bronchoscopy system is to assist the physician in navigating the real bronchoscope through the subject's airway tree so that they follow the preplanned airway route r defined in virtual space. A vital underpinning of assisted bronchoscopy systems is that guidance is accomplished by aligning the positions of the virtual and real bronchoscopes as they proceed along the guidance route toward the ROI in their respective spaces. This suggests that as the virtual bronchoscope moves along route r the real bronchoscope should be maneuvered so that it appears to be at the same location. In other words, during the live procedure, the bronchoscope's video stream should appear to stay approximately synchronized, or registered, with the virtual bronchoscope's VB view sequence along r, at certain view sites/poses Θⱼ along the guidance route; i.e.,

$${I}_{V}^{{Θ}_{j}^{′}} \approx {I}_{CT}^{{Θ}_{j}} , j = 1 , 2 , ... \qquad ( 9 )$$

where
I_{CT}^{Θ}^{ⱼ} is a VB view
Θⱼ≙virtual space pose of a view site on route r
I_{V}^{Θ′}^{ⱼ} is a frame in the bronchoscope's video stream
Θⱼ′≙A real space pose approximately corresponding to the same viewpoint as Θⱼ.

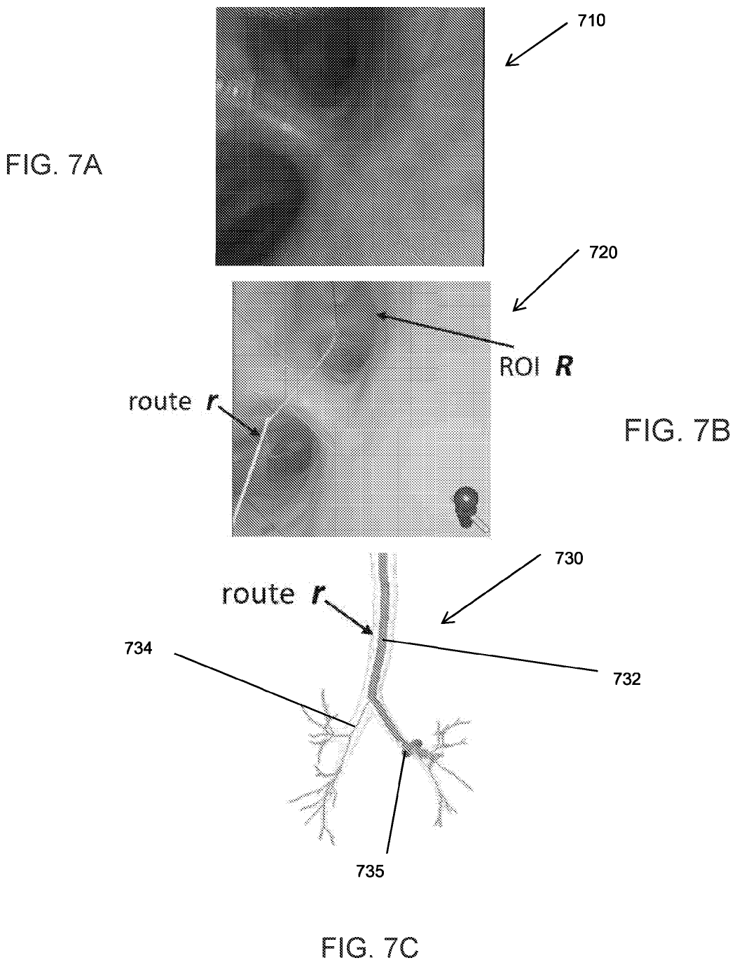

Importantly, at the conclusion of guidance during final confirmation and/or localization, the bronchoscope and VB are registered at the destination view site v_{D}. FIGS. 7A-7C illustrate many of the concepts discussed above for assisted bronchoscopy. More specifically FIGS. 7A-7C illustrate examples of virtual and real space poses registered at a view site of a guidance route. FIG. 7A depicts a still image 710 captured from a live bronchoscopic video frame I_{V}^{Θ′}^{ⱼ}. FIG. 7B depicts a VB view 720, I_{CT}^{Θ}^{ⱼ}, generated at virtual space pose Θⱼ for a view site on route r. FIG. 7C depicts a 3D CT-based airway tree showing the preplanned guidance route r 732 to ROI R and centerlines 734. The cylindrical icon 735 indicate a current virtual space pose Θⱼ of the virtual bronchoscope.

Returning to the planning process portion of the method, the method continues as a 3D transformation T is now computed that specifies how each point I_{TLC}(x, y, z) maps to a corresponding point I_{FRC}(x, y, z). By deriving transformation T, the means is available for relating the high-quality reference-standard TLC planning data to the potentially less complete data observed at FRC.

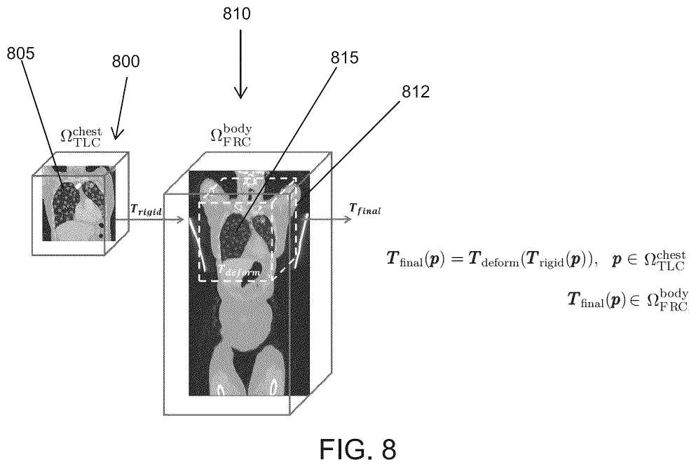

The following discussion assumes CT scan I_{FRC} is derived from a whole-body PET/CT study. Hence, 3D digital image I_{FRC} resides in the domain Ω_{FRC}^{body}⊆ [symbol] ³, which delineates the 3D CT-based virtual space relative to the FRC scan. Likewise, 3D digital image I_{TLC} resides in the domain Ω_{TLC}^{chest}⊆ [symbol] ³, which delineates the 3D CT-based virtual space relative to the TLC scan. FIG. 8 depicts these two spaces and coordinate systems. Note that the 3D spatial resolution of the two CT scans shown in FIGS. 8A and 8B differ in (Δx, Δy, Δz). In addition, the origins O of the two scans in their respective spaces differ.

Despite these differences, it is straightforward to establish point correspondences between the two spaces via deformable image registration. The basic approach has been applied to registering inflated and deflated 3D cone beam CT images and to the deformable registration of whole-body 3D PET scans to 3D chest CT scans. Notably, if CT scans, I_{TLC} and I_{FRC}, were collected with the same high-resolution chest CT scanner, then the spatial resolutions of the two CT scans would be the same and only their origins would differ. Hence, the transformation process for this simpler situation readily follows from the current discussion.

Computation of the desired 3D transformation T involves finding the optimal transformation T=T_{O} that enables alignment of I_{TLC} and I_{FRC}. It requires solving the optimization problem

$${T}_{o} = arg \{ {min}_{T} C ( {I}_{TLC} ( p ) ) , {I}_{FRC} ( T ( p ) ) \} , ∀ p ∈ {Ω}_{TLC}^{chest} . \qquad ( 10 )$$

where C(·, ·) is a cost function and T(·) is a transformation that maps each point p=(x,y,z)∈Ω_{TLC}^{chest}. Thus for each point p∈Ω_{TLC}^{chest},

$$\hat{p} = {T}_{o} ( p ) .$$

where p̅∈Ω_{FRC}^{body} and
I_{TLC}(p) corresponds to I_{FRC}(p̅).

Two well-known approaches exist for solving problem (10).

The first approach entails a traditional two-step optimization process that seeks to derive a transformation of the form

$${T}_{final} = {T}_{deform} ( {T}_{rigid} ) \qquad ( 11 )$$

where T_{rigid} and T_{deform} are rigid and deformable transformations, respectively, i.e., for each p∈Ω_{TLC}^{chest},

$$\tilde{p} = {T}_{final} ( p ) = {T}_{deform} ( {T}_{rigid} ( p ) )$$

where p̅∈Ω_{FRC}^{body}.

FIG. 8 gives a schematic diagram of the transformation T_{final} between Ω_{TLC}^{chest} and Ω_{FRC}^{body}. More specifically, FIG. 8 depicts a chest space 800, Ω_{TLC}^{chest}, and a whole-body space 810, Ω_{FRC}^{body}, for 3D transformation. The TLC-based space 800, Ω_{TLC}^{chest} is mapped into the FRC-based space 810, Ω_{FRC}^{body}, represented as dotted cube 812, through transformation T_{final}. Points, or dots, 805 in Ω_{TLC}^{chest} are mapped to corresponding points, or dots, 815 in Ω_{FRC}^{body}. Deriving T_{final} involves two steps: (1) initial alignment; and (2) deformable registration.

The first step of initial alignment derives the requisite rigid transformation T_{rigid} that performs a simple 3D translation and rotation to roughly align I_{TLC} with I_{FRC}. It exploits the idea that the trachea and the left and right main bronchi are essentially bony rigid structures whose shapes do not change from TLC to FRC. In particular, by finding the transformation T_{rigid} that aligns these major airway structures in the two CT scans, the desired T_{rigid} can be obtained. The method for finding T_{rigid} involves the following three steps.

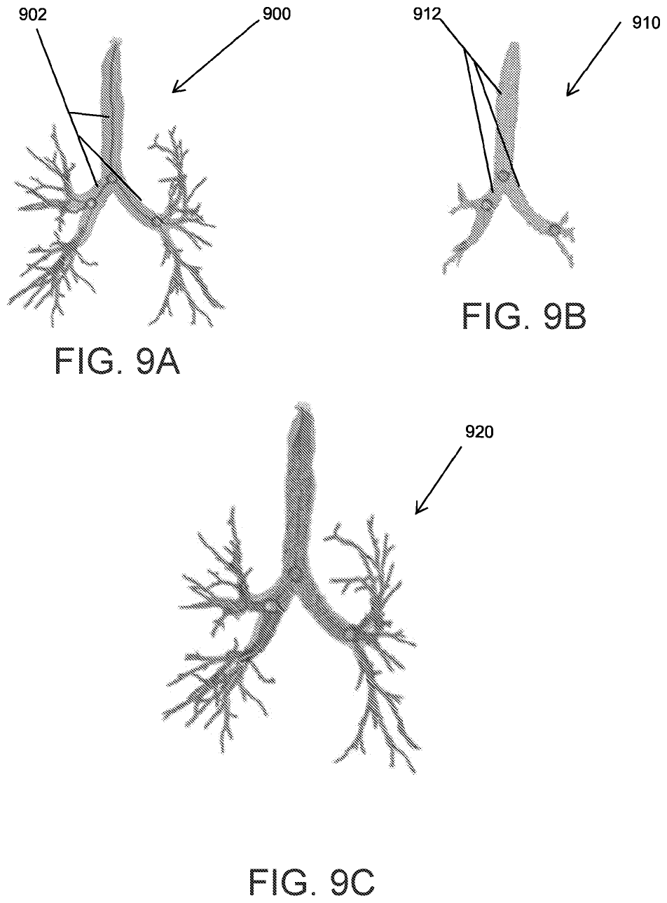

First, using the previously computed segmented airway trees associated with the two CT scans, the trachea b_{trachea} and left and right main bronchi, b_{left} and b_{right} are identified while omitting other airway branches. This gives M^{chest} and M^{body}, which represent the main-branch airway tree surface points of I^{chest} and I^{body}, respectively. FIGS. 9A and 9B depict example trees found for an example CT scan pair. More specifically, FIGS. 9A and 9C depict a registration of TLC- and FRC-based airway trees. FIG. 9A depicts a segmented airway tree 900 and centerlines 900 from I_{TLC}. Three main airway branches 902, trachea b_{trachea}, left main bronchus b_{left}, and right main bronchus b_{right}, are highlighted. FIG. 9B depicts the segmented airway tree 910 and centerlines from I_{FRC}. The three main airway branches 912 are once again highlighted.

Second, one thousand (1000) points are randomly selected from FRC surface M^{body}. However, in various instances, more or less than 1000 points can be selected. Next, for each selected FRC point, an iterative closest point registration method is applied to find the closes point in M^{chest} corresponding to the FRC point.

Thirdly, given the corresponding point pairs, the least-squares fitting is used to estimate a desired rigid transformation T_{rigid}.

FIG. 9C depicts the result 920 of applying the derived T_{rigid} to align the two trees 900, 910, while FIG. 2 depicts results after applying T_{rigid} to I_{TLC}. While the initial alignment provided by T_{rigid} makes much progress in the transformation process, substantial errors still exist between I_{TLC}(p) and I_{FRC}(T_{rigid}(p)) at many points p∈Ω_{TLC}^{chest}. Notably, even though the lower-resolution I_{FRC} provide a substantially reduced tree as compared to I_{TLC}, more than sufficient surface information exists for performing the initial coarse alignment.

The second step of deriving T_{final} involves deformable registration that alleviates much of the remaining error. Deformable registration involves a 3D intensity-based optimization process involving two images: (1) a fixed image I_{fixed}(p)=I_{FRC}(p); and (2) a moving image I_{moving}(p)=I_{TLC}(T_{rigid}(p)), ∀p∈Ω_{TLC}^{chest}. The goal is to find a spatially varying transformation function T_{deform}=T_{μ} that gives the best match between I_{fixed}(p) and I_{moving}(T_{μ}(p)) for all points p∈Ω_{TLC}^{chest}. To find this transformation, a B-spline-based deformation model is employed constituting the Elastix optimization framework. This entails solving the optimization problem

$$μ *= arg \{ {min}_{μ} C ( {I}_{fixed} ( p ) , {I}_{moving} ( {T}_{μ} ( p ) ) ) \} , ∀ p ∈ {Ω}_{TLC}^{chest} , \qquad ( 12 )$$

where Tμ is a 3D parametric transformation function defined by parameter set μ* and C is a cost function that measures the intensity similarity between two images. Here, a lower value indicates a greater similarity. In practice, to give more pertinent results in the region that matters—namely, the thoracic cavity—the space considered in (12) is reduced to points p∈Ω_{TLC}^{thoracic}, where Ω_{TLC}^{thoracic} is the restriction of space Ω_{TLC}^{chest} to points inside the segmented thoracic cavity.

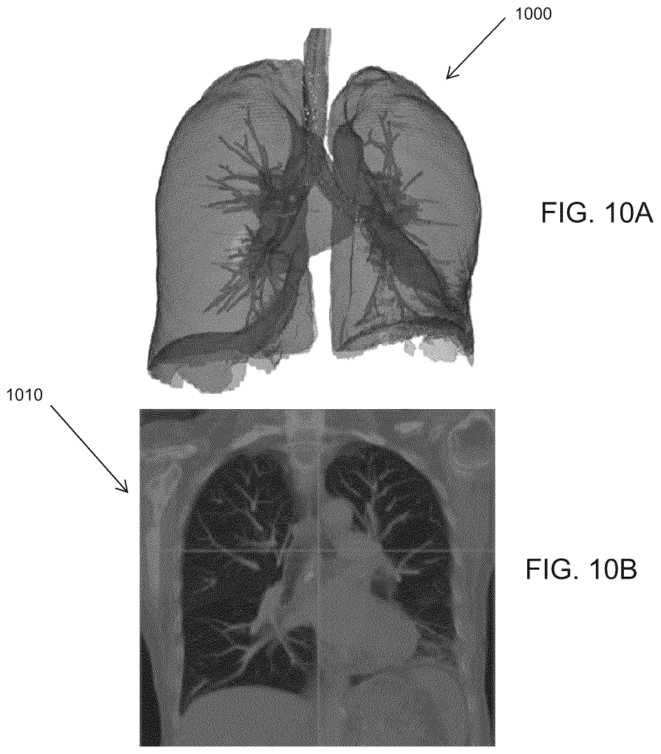

FIGS. 10A and 10B depict results of applying the complete transformation Tfinal after applying Tdeform to the result of FIG. 2. FIGS. 10A and 10B depict the results after rigid alignment and deformable registration. FIG. 10A depicts fully registered volume renderings 1000 of the organs for a TLC volume and an FRC volume. FIG. 10B depicts a coronal front-to-back slab view 1010 depicting the volume differences between the fully-registered TLC volume and FRC volume. Also, derived transformation T is used to map all TLC-based components of the anatomical chest model (segmented airway tree, endoluminal surfaces, center-lines, ROI R, etc.) into FRC-based virtual space.

An alternative approach for solving optimization problem (10) to find 3D transformation T involves using deep learning techniques. Such methods have been applied to image registration in many domains, including the registration of CT image volumes to cone beam CT and the registration of 3D CT cardiac volumes at different time points. For chest CT, unsupervised learning methods for deformable registration of 3D volumetric images at different time points in the breathing cycle have been proposed. For these methods, the inputs are again a fixed image I_{fixed} and a moving image I_{moving}, which pass through a deep learning framework to derive the 3D displacement field T which relates the correspondences between the two images. Results show that the methods perform successfully, particularly in relation to known packages such as Elastix. An added advantage of these methods is that they tend to be extremely fast computationally.

After deriving 3D transformation T, the planning process of the method proceeds by deriving a TLC-based primary guidance route rₚ, an FRC-based final guidance route r_{f}, and associated guidance cues for each route.

To begin, using CT scan I_{TLC}, primary guidance route rₚ is computed. The route computation method draws on the following inputs: (1) bronchoscope specifications (d_{B}, λ_{B}, Δ_{B}), where d_{B} and λ_{B} indicate the diameter and length of the bronchoscope tip, respectively, and Δ_{B} denotes the maximum bending angle of the bronchoscope tip when it is articulated; (2) biopsy needle, or rEBUS probe, length λ_{N}, which also indicates the maximum distance that the supplemental device can be extended out of the bronchoscope tip; (3) a 3D cone, whose axis is the needle/probe and whose radius is defined by angle θ_{cone}, given by

$$sin θ cone = \frac{r}{λ}$$

where λ≥λ_{N} is the length in mm that the needle/probe is extended from the bronchoscope tip and r is the resulting cone radius. A feasible airway guidance route terminates with a destination site v_{D} that avoids occluding blood vessels. This condition is met by a route if the cone's 3D volume does not intersect nearby vessels when location is reached v_{D}. The cone also guards against the uncertainty in accuracy that the needle, or probe, can be positioned, any imperfections in the definition of ROI R, and/or image resolution approximations; and/or (4) anatomical chest model quantities (a) airway branch lengths l_{b}_{ⱼ} and diameters d_{b}_{ⱼ} for all bⱼ∈B and branch angles between successive branches, (b) airway surface data (c), ROIs corresponding to occluding obstacles, including possibly the aorta, pulmonary artery, lungs, and/or other vasculatures, and/or (d) ROI R.

The goal is to derive a primary route rₚ of the form (6) that is both feasible and optimal with respect to two feasibility constraints and one optimality constraint. The first feasibility constraint is defined as the bronchoscope can fit within the airways constituting route rₚ, relative to the constraints imposed by the bronchoscope and needle/probe specifications (d_{B}, λ_{B}, Δ_{B}, λ_{N}) and/or known airway branch measurements and surface data, obstacle locations and shapes, and ROI R. The second feasibility constraint is defined as the device either does not puncture occluding anatomical structures (needle used) or does not have its view of the ROI obscured (probe used), relative to the constraints imposed by the cone and/or locations and/or shapes of the known obstacles, such as the aorta, etc., for example. The optimality constraint is defined as the destination pose v_{D} enables a maximal tissue sample relative to other possible poses.

The above requires a search for the optimal route rₚ=rₒ within tree structure T that enables either the maximum tissue sample of R (if a needle is used) or maximal 1-D cross-sectional view of R (if a probe is used) per the relation:

$${r}_{o} = arg \{ {max}_{r ∈ {S}_{f}} {E}_{r} ( D ( {d}_{B} , {λ}_{B} , {Δ}_{B} , {λ}_{N} ) ) \} , \qquad ( 13 )$$

where S_{f} is the set of feasible routes per the feasibility constraints and D(·) is a function denoting ROI tissue sample size (needle used) or 1-D ROI cross-section width (probe used). Lastly, E (D (·)) is a function that recognizes that sampling at the location x of v_{D} will entail some uncertainty into how closely the physician/robot will be able to position the needle/probe in direction d. An efficient implementation of optimization problem (13) can be performed on a computer.

For the purposes of this disclosure, the final derived primary guidance route rₚ takes the following modified form of (5-6):

$${r}_{p} = \{ {b}_{1} , {b}_{2} , … , {b}_{j} , … , {b}_{N - 1} , {b}_{N} , s \} \qquad ( 14 )$$

$$\begin{matrix}= \{ {v}_{1} , … , {v}_{1 , ω 1} , {v}_{2 , 2} , … , {v}_{2 , ω 2} , {v}_{3 , 2} , … , {v}_{3 , ω 2} , … , \\ \underbrace{{v}_{N - 1 , {ω}_{N - 1}} , {v}_{N , 2} , … , {v}_{N , {ω}_{N}}}_{{b}_{N}} , {v}_{N + 1 , 2} , … , {v}_{D} \}\end{matrix} \qquad ( 15 )$$

$$\begin{matrix}= \{ {v}_{1} , … , {v}_{1 , ω 1} , {v}_{2 , 2} , … , {v}_{2 , ω 2} , {v}_{3 , 2} , … , {v}_{3 , ω 2} , … , \\ \underbrace{{v}_{L} , {v}_{N , 2} , … , {v}_{N , {ω}_{N}}}_{{b}_{N}} , \underbrace{{v}_{N + 1 , 2} , … , {v}_{D}}_{s } \} .\end{matrix} \qquad ( 16 )$$

Relation (16) highlights a major added novel element in the designation of a localization site v_{L}, which is also the endpoint v_{N-1}, ω_{N-1} of branch b_{N-1} and the first view site v_{N, 1} of branch b_{N}. (Recall that v_{N, 1}=v_{N-1}, ω_{N-1}.) The localization view site v_{L} serves as the bridge for creating the final guidance route r_{f} residing in FRC-based virtual space. In particular, it signifies when guidance shifts from the primary guidance route rₚ to the final guidance route r_{f}—for this point onward, any remaining navigation followed by final ROI confirmation/localization is done relative to FRC-based virtual space.

In practice for this disclosure, v_{L} is designated as the branch endpoint in rₚ closest to destination site v_{D} whose cumulative Euclidean distance to v_{D} is ≥d_{L} mm, where d_{L} is a user-selected parameter in the 2 to 4 cm range for bronchoscopy. Most importantly, d_{L} is selected so that v_{L} is the endpoint of a branch that is also definitely visible in FRC-based virtual space. Note, without loss of generality, that (16) assumes that v_{L} ends branch b_{N-1}, but it can be any branch bb, b≤N, depending on d_{L}. The definition of r_{f} provided herein will clarify this point.

Given primary guidance route rₚ, it is a simple matter to derive associate guidance cues. The purpose of these cues is to direct the clinician and/or robot on how to maneuver and deploy the bronchoscope and/or supplemental device along a given route during a live procedure. Cues can be derived for each phase of bronchoscopy guidance.

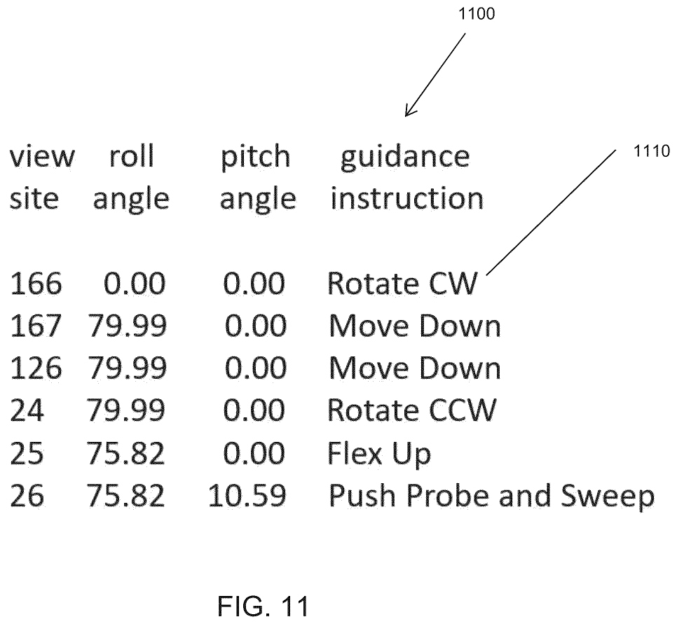

For the navigation phase (view sites of rₚ>d_{local} mm from v_{D}), a cue is derived at each airway bifurcation, which suggests that the bronchoscope either be moved up or down or rotated clockwise or counterclockwise. For the confirmation and/or localization phase (view sites of rₚ<d_{local} mm from v_{D}), the remaining cues suggest how to deploy the supplemental device. These cues can include a final bronchoscope movement and rotation, to position the bronchoscope for device deployment, and cues for using the device (e.g., push probe and sweep). Note that the planning process automatically determines distance d_{local}, based on the previously discussed feasibility and optimality constraints. Also, d_{local}≤d_{L}. FIG. 11 provides an example excerpt 1100 of a series of cues 1110 derived for a guided rEBUS procedure.

Final guidance route r_{f} residing in FRC-based virtual space is now derived. To do this, 3D transformation T is applied to map all view sites constituting rₚ from (16) into the space delineated by I_{FRC}. It is important to ensure all transformed view sites are forced to be within the confines of the transformed FRC-based segmented airway tree and surfaces. This is accomplished by moving mapped view sites slightly such that they stay within the airway tree's endoluminal surface. Lastly, all mapped view sites including the destination site abides by the feasibility and optimality constraints observed by rₚ. Because these constraints may make it overly difficult to find a route sufficiently close to ROI r, a secondary option for r_{f} that relaxes these constraints can also be derived. Overall, this gives

$${r}_{f} = \{ {\hat{b}}_{1} , {\hat{b}}_{2} , … , {\hat{b}}_{j} , … , {\hat{b}}_{b - 1} , {\hat{b}}_{b} , \hat{s} \} \qquad ( 17 )$$

$$= \{ {\hat{v}}_{1} , … , {\hat{v}}_{1 , ω 1} , {\hat{v}}_{2 , 2} , … , {\hat{v}}_{2 , ω 2} , {\hat{v}}_{3 , 2} , … , {\hat{v}}_{3 , ω 3} , … , \qquad ( 18 )$$

$$\underbrace{{\hat{v}}_{b , 1} , {\hat{v}}_{b , 2} , … , {\hat{v}}_{b , {ω}_{b}}}_{ {\hat{b}}_{b}} , \underbrace{{\hat{v}}_{b + 1 , 2} , … , {\hat{v}}_{D}}_{\hat{s} } \} .$$

where branches b̅ and view sites \hat{v} are situated in FRC-based virtual space. Note that the view sites v and v̅ constituting rₚ and r_{f}, respectively, all differ in general as they reside in different spaces. Also, comparing (16) and (18), the number of complete airway branches N constituting rₚ differs in general from the number of complete branches b constituting r_{f}. In particular, b≤N. This is exemplified by FIG. 4. This situation arises because smaller terminal airways may become closed or excessively narrowed when the lungs are at FRC—these airways, however, still exist in the physical lungs at FRC. Notably, v_{L} of rₚ can be the endpoint of a branch bⱼ in (14) visited before branch b_{N}; i.e., v_{L}∉b_{N} as suggested by (16), but instead is an element of bⱼ, j<N.

Elaborating on the observations above, because the intrinsic hierarchy of the airway tree's branching structure is maintained whether the lungs are at TLC or FRC, the following branch correspondences between rₚ and r_{f} exist:

$$( 19 )$$

$${b}_{1} \to {\hat{b}}_{1}$$

$${b}_{2} \to {\hat{b}}_{2}$$

$$⋮ \to ⋮$$

$${b}_{b} \to {\hat{b}}_{b}$$

Also, per (19), TLC-to-FRC correspondences exist between all view sites constituting the first b branches of the two routes, i.e.,

$${v}_{α , {ω}_{2}} \to {\hat{v}}_{α , {ω}_{j}} \qquad ( 20 )$$

for all view-site indices a arising for a particular branch pair (bⱼ, \hat{b}ⱼ), j=1, . . . , b.

In addition, a special correspondence between the location of localization site V_{L}∈rₚ and the corresponding view site in r_{f} is noted; i.e.,

$${v}_{L} \to {\hat{v}}_{c , {ω}_{c}} ≡ {\hat{v}}_{L} \qquad ( 21 )$$

for some branch \hat{b}_{c}∈r_{f}, c≤b, having branch endpoint \hat{v}_{c,ω}_{ₑ}. Correspondence (21) is pivotal during later live guidance, as it signals when to switch guidance from rₚ to the final guidance route r_{f}. Notably, r_{f} does not employ a localization view site v_{L} per se as required by rₚ.

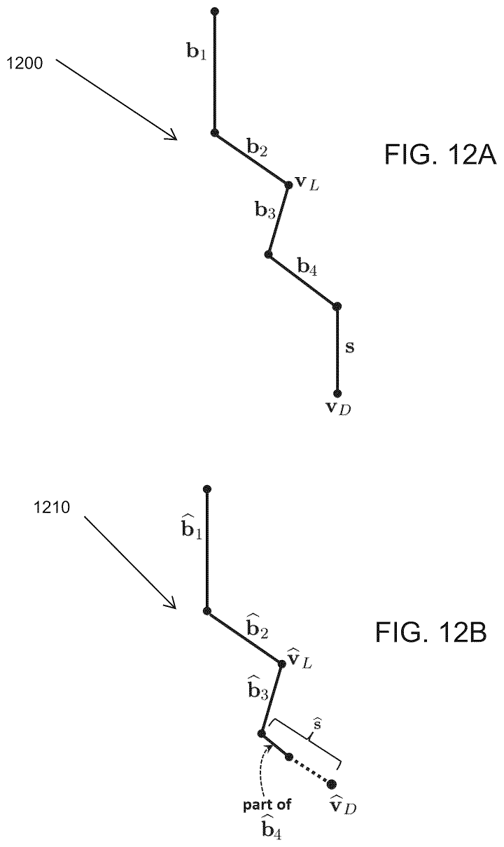

FIGS. 12A and 12B schematically compare the structures of rₚ and r_{f}. Stated another way, FIGS. 12A and 12B schematically compare the possible routes rₚ and r_{f}. FIG. 12A represents an example primary guidance route rₚ 1200 while FIG. 12B represents an example final guidance route r_{f} 1210. In the depicted example, TLC-based branches b₁, b₂, and b₃ correspond to FRC-based branches \hat{b}₁, \hat{b}₂, and \hat{b}₃, respectively, per (19), while poses v_{L} and \hat{v}_{L} correspond to the branch endpoints of the second branches of each route. Lastly, destination poses v_{D} and \hat{v}_{D} are at different locations in general.

Per FIGS. 12A and 12B and the discussion herein, route r_{f} will in general omit a concluding part of route rₚ but have a view site (and corresponding branch) corresponding to v_{L} per (21). Also, the final destination v_{D} for r_{f}, will differ as a rule from the TLC-based v_{D}. Related to this point, route r_{f} will generally be shorter than rₚ and terminate at a final destination further from the ROI than rₚ. Note again, however, that the airway branches of rₚ that are not members of r_{f} still exist in the real airway tree.

As a final planning operation, guidance cues for the navigation and confirmation/localization phases are derived for r_{f}, as completed earlier for rₚ.

Notably, the CT scan and planning data used for FRC-based virtual space is the TLC-to-FRC transformed version of I_{TLC}, not I_{FRC}. As FIGS. 3A-3C show, the TLC CT scan gives the most extensive airway tree, with the TLC CT scan mapped to FRC space giving a more extensive tree than the raw input FRC-based CT scan. In terms of lung ventilation level, the TLC-to-FRC transformed CT scan and the raw FRC CT scan are mapped to the same 3D FRC-based space, yet the TLC-to-FRC is able to show more airway tree structure, which can prove to be very useful during live guidance—hence, the planning/guidance operations are based on this scan. As discussed herein, the raw FRC scan is also available during guided bronchoscopy for reference.

Picking up from the discussion of FIGS. 12A and 12B, note that as the lung state changes from TLC to FRC, certain airway branches further from v_{D} than branch b_{N}∈rₚ in (16) could become excessively compressed or blocked, including those before the b^{th} branch constituting final guidance route r_{f} in (18). In fact, phenomena such as mucus or atelectasis could also make this happen at the time of the live procedure regardless of the chest's state. Hence, in such circumstances, (19) will not hold up to branch index b and r_{f} as given by (18) will not be feasible. This could halt guidance well before reaching the destination. A solution for circumventing this situation would be to call for a real-time change during the procedure in the airway guidance route before reaching localization site v_{L}∈b_{N} of rₚ. This requires more available alternative routes reaching ROI R than just route r_{f}.

In various instances, or alternative methods, this flexibility is provided by incorporating the concept of back-up guidance routes. A series of up to n≤N back-up guidance routes rⁿ, n=1, . . . . N, residing in TLC space of the form

$${r}^{n} = \begin{matrix}\{ {b}_{1} , … , {b}_{n} , & \underbrace{{b}_{n + 1}^{′} , … , {b}_{d}^{′} , {s}^{′} \}}_{new portion }\end{matrix} \qquad ( 22 )$$

$$= \{ \begin{matrix}{v}_{1} , … , {v}_{n , {ω}_{n}} , {v}_{n + 1 , 2}^{′} , … , {v}_{L}^{n} , … , \\ {v}_{d , {ω}_{d}}^{′} , {v}_{d + 1 , 2}^{′} , … , {v}_{D}^{n}\end{matrix} \} \qquad ( 23 )$$

$$= \{ \begin{matrix}{v}_{1} , … , \underbrace{{v}_{n + 1 , 1}^{′} , {v}_{n + 1 , 2}^{′} , … , {v}_{L}^{n} , … , {v}_{d , {ω}_{d}}^{′}}_{ new branches } , \\ \underbrace{{v}_{d + 1 , 2}^{′} , … , {v}_{D}^{n}}_{ {s}^{′}}\end{matrix} \} .$$

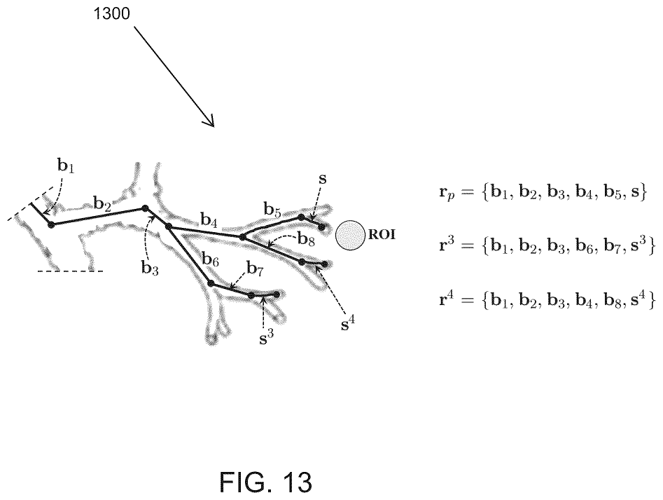

including the first n branches of primary guidance route rₚ of (14) along with new branches bₙ₊₁′ through b_{d}′ in B, new localization site v_{L}ⁿ, new destination site v_{D}ⁿ, and new segment s′. The value of d can differ for each back up guidance route. FIG. 13 depicts a schematic 1300 of exemplary back-up guidance routes. For the pictured ROI and nearby airway tree section, derived primary guidance route rₚ and back-up routes, r³ and r⁴, are depicted. Following (22), branches b₆ and b₇ of back-up route r³ correspond to branches, b₄′ and b₅′, respectively, while branch b₈ of back-up route r⁴ corresponds to branch b₅′. Also, the segments s³ and s⁴ correspond to segment s′ for back-up routes r³ and r⁴, respectively.

Relation (17) for r_{f}, however, indicates that only back-up routes up to branch index n=b, b≤N, at most will be needed; i.e., only back-up routes rⁿ, n=1, . . . b, need actually be computed. These routes are computed via the same optimization process used to derive rₚ with the added constraint that the (n+1)^{th} branch bₙ₊₁′ constituting rⁿ cannot equal bₙ₊₁∈rₚ.

Next, each back-up route rⁿ requires an associated final back-up route r_{f}ⁿ FRC-based virtual space, similar to how primary guidance route rₚ has an associated FRC-based final guidance route r_{f} per (17-18). The final back-up routes are again computed as before, with final back-up route r_{f}ⁿ taking the form

$$\begin{matrix}{r}_{f}^{n} = \{ {\hat{b}}_{1} , … , {\hat{b}}_{c - 1} , {\hat{b}}_{c} , \hat{s} \} \\ = {\{ {\hat{v}}_{1} , … , {\hat{v}}_{e , 1}^{′} , …}_{{︸}_{{\hat{b}}_{n}^{′}}} , {\hat{v}}_{e , {ω}_{a}}^{′} , \underbrace{{v}_{e + 1 , 2}^{′} , … , {v}_{D}}_{ {s}^{′} }\end{matrix} , , \qquad ( 24 )$$

where once again branches b̅ and view sites v̅ are situated in FRC-based virtual space. Also, the number of complete airway branches d constituting back-up route rⁿ differs in general from the number of complete branches e constituting r_{f}ⁿ; i.e., e≤d. In addition, a special correspondence between the location of localization site v_{L}ⁿεrⁿ and a corresponding view site in r_{f}ⁿ exists; i.e.,

$${v}_{L}^{n} \to {\hat{v}}_{f , {ω}_{f}} \qquad ( 25 )$$

for some branch \hat{b}_{f}∈r_{f}ⁿ, f≤e, having branch endpoint \hat{v}_{f,ω}_{_{f}}. Correspondence (25) is again pivotal during later live guidance, as it suggests when to switch from back-up route rⁿ to FRC-based route r_{f}ⁿ. If a view site \hat{v}_{f,ω}_{_{f}} satisfying (25) cannot be found, the final back-up guidance route r_{f}ⁿ cannot be defined. This in turn implies that back-up route rⁿ does not admit a feasible path to ROI R in FRC-based space. Hence, rⁿ is eliminated from the set of back-up routes retained for later guided bronchoscopy and no back-up option will exist for the nth branch along primary guidance route rₚ. Conversely, additional back-up guidance routes arising at a bifurcation n̅>n for a given back-up route rⁿ can also be derived. However, few such additional back-up routes are likely feasible, given the finite branching complexity of the airway tree.

As another consideration, it is possible that for some n₁ in the range 1≤n₁≤b, no candidate back-up route rⁿ⁻¹ satisfies the constraints required by the optimization process; e.g., any existing child branch of bₙ_{₁}_{-1∈r}_{ₚ} that is not equal to bₙ_{₁}∈rₚ ends in a “dead end” not leading to ROI R. Such situations also reduce the total number of back-up routes available for later bronchoscopy.

Analogous to (19) and (21), correspondence between branches for back-up route rⁿ and associated final back-up route r_{f}ⁿ exist as follows:

$$( 26 )$$

$${b}_{1} \to {\hat{b}}_{1}$$

$$⋮ \to ⋮$$

$${b}_{e} \to {\hat{b}}_{e}$$

Also, TLC-to-FRC correspondences exist between all view sites constituting the first e branches of the two routes; i.e.,

$${v}_{α , {ω}_{j}} \to {\hat{v}}_{α , {ω}_{j}} \qquad ( 27 )$$

for all view-site indices α arising for a particular branch pair (bⱼ, \hat{b}ⱼ), j=1, . . . , e.

Notably, the one-to-one TLC-to-FRC correspondences as specified by (19-21) and/or (26-27), which exploit the invariant 3D topology and hierarchy of the airway tree, are a major part of the procedure plan.

Lastly, as done earlier for rₚ and r_{f}, guidance cues are derived and associated with each back-up guidance route and final back-up guidance route. In addition, supplemental guidance cues are created to help signal the need for switching to a back-up guidance route at any bifurcation where a route rⁿ is available. This can be signaled by the general instruction: “Alternate route is available at this juncture.” Alternatively, if the system determined earlier during preplanning that the FRC-based shape of the airways will become excessively narrow during the live procedure, the following instruction can be issued: “Next airway on route is predicted to be too narrow; alternate route suggested.” Additional cues can also be produced to point out the distance that a destination pose is from ROI R, such as: “Guidance route can only get within 33 mm of the ROI.”

After deriving the procedure plan, the method continues by a performance of a live guided bronchoscopy. Such a guided bronchoscopy can now take place in an operating room using the assisted bronchoscopy system's guidance computer. Initial set-up in the operating, or other suitable procedure, room includes the following steps.

Firstly, the procedure plan is loaded into the guidance computer, including all CT scan data, anatomical chest model data, preplanned routes, and associated guidance cues. The bronchoscope hardware is then interfaced to the system. In particular, bronchoscope video outputs, including video from supplemental devices (e.g., rEBUS) are interfaced with the system. The guidance computer's display is then initialized for ROI R. Such initialization includes at least (1) a 3D surface rendering of the airway tree, airway centerlines, active guidance route, and ROI R; (2) an endoluminal VB view, with airway centerlines, active guidance route, and ROI R; (3) live bronchoscope video stream views; and/or (4) a display for text-based guidance cues and distance measurements.

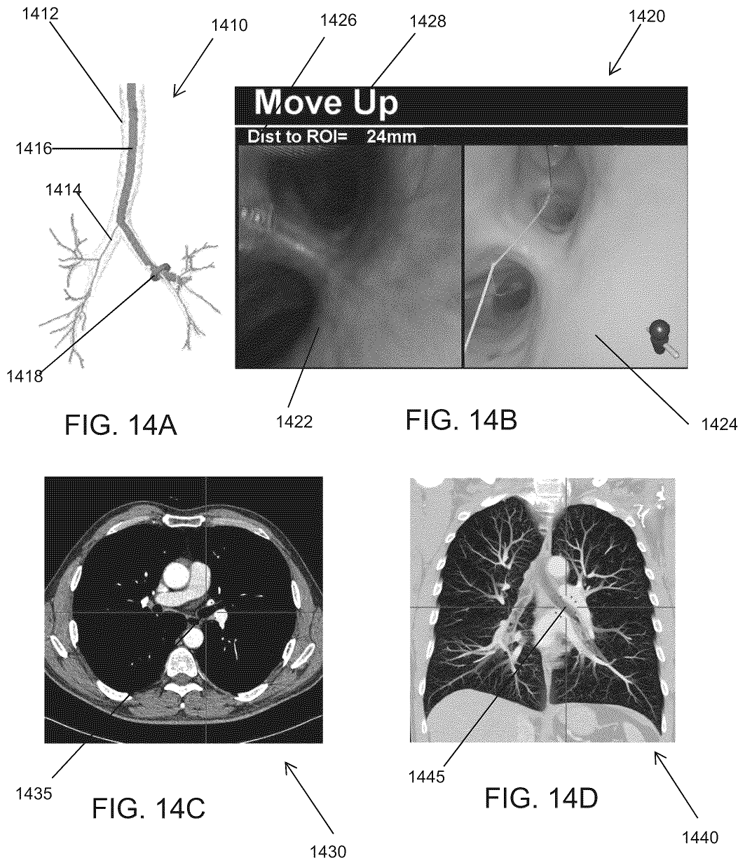

FIGS. 14A-14D depict examples of the basic elements of the guidance computer's display for assisted bronchoscopy. FIG. 14A depicts a 3D surface rendering 1410 of an airway tree 1412 of the subject with centerlines 1414, an active guidance route 1416, and an ROI 1418. FIG. 14B depicts an integrated viewer 1420 depicting a live bronchoscopic video 1422, a VB view 1424 for the guidance system's current view site depicting the guidance route and ROI, a remaining distance 1426 to navigate toward the ROI, and a guidance cue 1428. FIG. 14C depicts a 2D axial-plane CT section 1430, and FIG. 14D depicts a coronal slab rendering 1440. All views are synchronized to the current guidance system's current view site along the guidance route. For FIGS. 14C and 14D, the view side is indicated by cross hairs 1435, 1445, respectively.

In various instances, the display can additionally and/or alternatively include other 2D or 3D graphical visualization tools, adapted from previously developed multimodal assisted bronchoscopy systems, such as 2D PET/CT section viewers (axial, coronal, sagittal orientations), sliding thin-slab visualizations, tube viewer, airway plot tool, 2D CT-based projection tools, and/or video analysis tools.

After system initialization, the clinician now performs the guided bronchoscopy procedure, with a goal of maneuvering the bronchoscope to a final destination pose sufficiently close to R. When the pose is reached, the clinician can perform a desired examination.

To begin, the clinician inserts the bronchoscope to a reference point, typically near the main carina. Next, the system's active guidance route is initialized to the preplanned primary guidance route rₚ. The guidance system then initializes the scope insertion depth to d_{ID}=0. It also initializes the remaining distance to the ROI d_{ROI}, based on route rₚ.

During the guided procedure, either the clinician or robot (under clinician control) maneuvers the bronchoscope through the airways, based on the cues and graphical views provided by the guidance computer.

With the system and clinician synchronized at the initial reference point, live guidance toward ROI R now follows a three-stage protocol including: (1) navigating toward the ROI; (2) optionally switching to a back-up guidance route; and (3) finally confirming and/or localizing the ROI.

Navigation toward the ROI is guided by the principle that the guidance system's CT-based virtual space position and the bronchoscope's location in the chest should correspond to approximately the same physical 3D chest position at all times during the live procedure per (9). During navigation, the clinician and/or robot maneuvers the bronchoscope through the larger airways suggested by the active guidance route. The system provides guidance by the cues suggested at each juncture along the route and by the displayed visual views. In particular, the clinician can execute the suggested cues, such as “Move Up,” “Rotate Clockwise,” etc., in an attempt to position the bronchoscope at the same apparent position shown currently by the VB viewer. In addition, periodic precise registrations between the bronchoscope and guidance system virtual bronchoscope can be performed.

During navigation all display views can stay synchronized to the current view site and/or are constantly, or otherwise routinely, updated, with progress along the route noted on the 3D surface viewer. Also, the scope insertion depth dip and distance to ROI d_{ROI} can give continuous, or otherwise routine, updates on progress of the procedure. The clinician can monitor all system information to note progress. Notably, linked TLC-space and FRC-space versions of all graphical viewers can be available by either toggling the guidance space (TLC or FRC) or a particular viewer, with all active plan information given in the current active space.

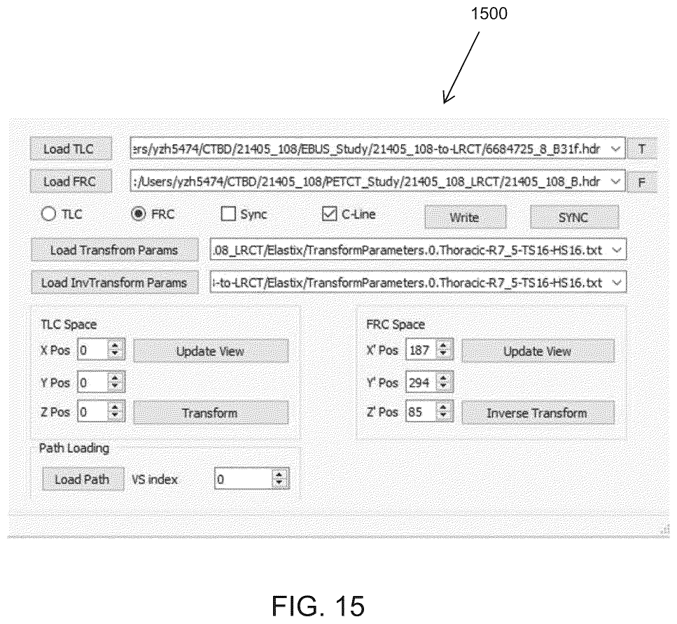

FIGS. 15-17D illustrate an example of this capability. A system, which integrates the methodologies described herein can be interfaced to an image-guided or robotics-assisted endoscopy system. The guidance system includes graphical tools that aide in deciding when to switch guidance routes as needed, signal an expected feasibility of moving forward on an active guidance route, and/or offer flexibility in visualizing the changing state of the airways during a live procedure.

In addition, system can also features the capability of switching between viewing graphical information in three synchronized spaces: 1) TLC-based graphical information, which embodies the ideal and most complete information on the airway tree anatomy; 2) TLC-to-FRC-based graphical information, which depicts the local airway tree anatomy in a way that is closer to the reality at the time of the live procedure; and/or 3) FRC-based graphical information, which depicts the original FRC-based CT data. For this purpose, graphical and software-control tools are available to enable simultaneous augmented reality viewing in these various 3D spaces.

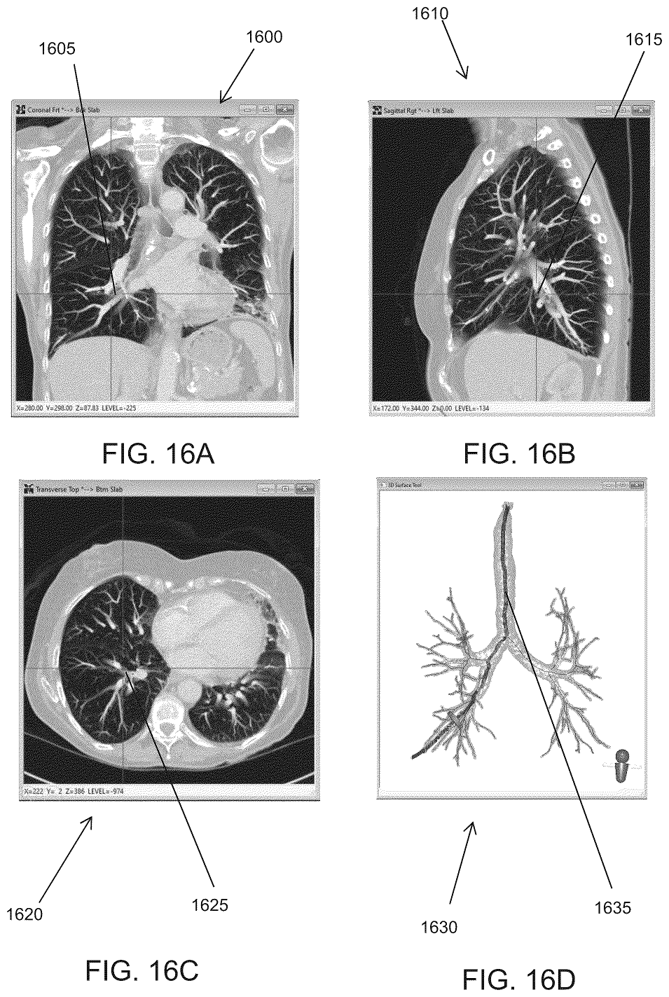

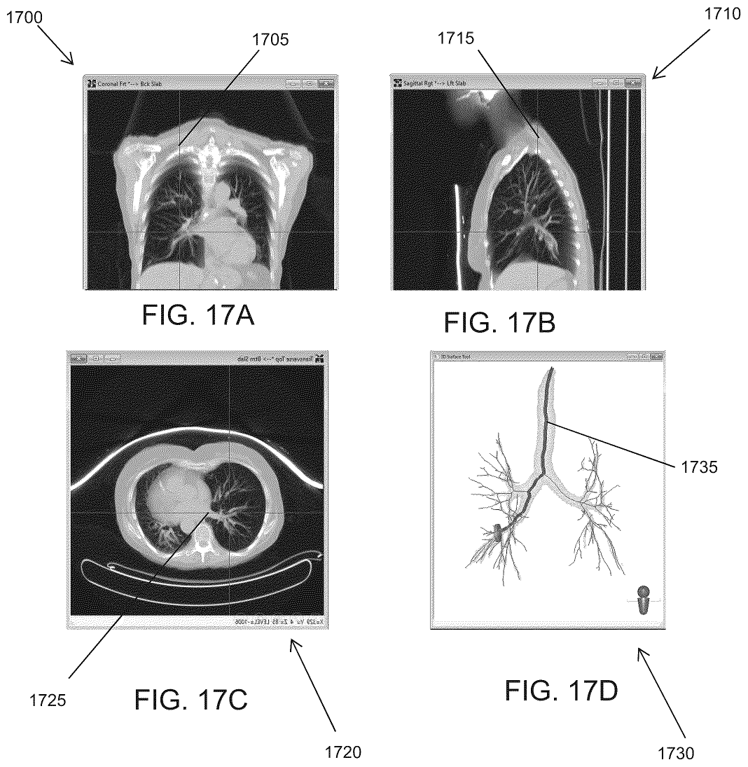

More specifically, FIG. 15 depicts a screen shot 1500 of a volume management tool of the system. The volume management tool is configured to coordinate synchronized views between the TLC-space and FRC-space views of FIGS. 16A-17D. FIGS. 16A-16D depict an example of live linkage of TLC-space and FRC-space views. The views draw on a high-resolution chest CT scan having dimensions 512×512×647, with voxel resolutions Δx=Δy=0.6 mm and Δz=0.5 mm. All views shown in FIGS. 16A-16D are in TLC space and synchronized to a view site at location (x. y. z)=(172, 298, 386) of path 14 which terminates in the right lower lobe of the subject lung. More specifically, FIG. 16A depicts a coronal depth-weighted slab view 1600 with cross hairs 1605 indicating the current view site. FIG. 16B depicts a sagittal depth-weighted slab view 1610 with cross hairs 1615 indicating the current view site. FIG. 16C depicts a transverse depth-weighted slab view 1620 with cross hairs 1625 indicating the current view site. FIG. 16D depicts a 3D surface rendered airway tree 1630 with a highlighted desired path 1635.

FIGS. 17A-17D depict an example of live linkage of TLC-space and FRC-space views. All views shown in FIGS. 17A-17D are in FRC space and synchronized to the view site shown in FIGS. 16A-16D. The views draw on a whole-body free-breathing CT scan, taken as part of a PET/CT study, having dimensions 512×512×279, with voxel resolutions Δx=Δy=0.88 mm and Δz=3.0 mm. More specifically, FIG. 17A depicts a coronal depth-weighted slab view 1700 with cross hairs 1705 indicating the current view site. FIG. 17B depicts a sagittal depth-weighted slab view 1710 with cross hairs 1715 indicating the current view site. FIG. 17C depicts a transverse depth-weighted slab view 1720 with cross hairs 1725 indicating the current view site. FIG. 17D depicts a 3D surface rendered airway tree 1730, depicting a version of TLC-space path 14 1735 mapped into FRC space.

FIGS. 16A-17D arise out of a subject study that included a high resolution breath-hold chest CT scan and a whole-body PET/CT study, where the PET/CT study supplies a lower-resolution free-breathing whole-body CT scan. For the example, an airway path was derived, as shown in FIG. 16D, that travels from the trachea to the terminus of a right lower lobe airway using the TLC-space chest CT scan. The path was mapped into FRC space, using the FRC-space whole-body CT scan as shown in FIG. 17D. To do this mapping, scope and airway diameter dimensions were disregarded; i.e., every view site of the TLC path was mapped into FRC space.

Referring back to FIG. 15, the depicted Volume Management Tool facilitates and manages the mapping and synchronization of all image data and path/route information between the TLC and FRC spaces. The tool illustrates how one can focus on TLC or FRC space, per the radio buttons, while the sync button enables synchronization of both spaces. In the example, the selected view site in TLC space at location (x, y, z)=(172,298,386) (FIGS. 16A-16D) maps to a corresponding view site in FRC space at location (x, y, z)=(187,294,85). Through the use of this volume synchronization and visualization feature, the clinician can notice how the airways and local anatomy change in shape between the initial TLC-planned procedure and the current free-breathing FRC live situation.

As navigation progresses from one airway branch to the next—i.e., without loss of generality, from airway branch bₙ₋₁ to airway branch bₙ—the system can optionally supply an alert for a possible alternate back-up route rⁿ, if available, to the ROI. As part of these alerts, the system may warn that an upcoming airway on the active guidance route is likely to be too narrow for bronchoscope passage and that it is advisable to switch navigation to back-up route rⁿ. Alternatively, the system may merely indicate that an alternate route exists.

The clinician is free to continue on the currently active guidance route or switch to a back-up route at any time. In various instances, the clinician may note that an upcoming airway bₙ₊₁ shown by the guidance display in reality appears to be blocked or narrowed in the live bronchoscopy video feed—for this situation, the clinician can switch the active guidance route to the available back-up route rⁿ, regardless of the guidance system's recommendation. All system display components can adjust to this change in route. In such instances, di remains unchanged, but d_{ROI} updates based on rⁿ. Navigation then proceeds as before.

When the guidance system reaches the active guidance route's localization pose V_{L}, usually 2 to 4 cm from the final destination and typically well into the periphery of the airway tree, the stage of the procedure is reached, whereby the impact of CTBD can become substantial. To help mitigate this issue, the system now switches the active guidance route to the complementary final guidance route situated in FRC-based virtual space. The final guidance route can be r_{f} or r_{f}ⁿ depending on if the active guidance route is rₚ or a back-up route rⁿ, respectively.

Upon making all system display components and distance d_{ROI} become updated within FRC-based space. The switch to FRC-based space will give a truer picture of the expected anatomy during tidal breathing and will provide a more accurate path to the target ROI.

Typically, upon transitioning to this stage, some remaining navigation is required, where d_{ROI}>d_{local}. The remaining navigation operations proceed as in the navigation stage. At last, the final cues, geared toward maneuvering the bronchoscope to its final position and then toward deploying the supplemental device are issued (bronchoscope≤d_{local} mm from the ROI). These cues enable the clinician and/or robot to localize the endoscope at the final destination pose v_{D} near the ROI, until the 3D positions of the virtual bronchoscope and real bronchoscope are synchronized near the optimal pose for examining the ROI. Also, at any time during final confirmation/localization, the reference TLC-based virtual space data is available and synchronized to whichever route is currently the active guidance route. Such information could be useful for example for visualizing an airway that is unexpectedly open or for noting a bronchus sign near the ROI.

Notably, it could be advantageous to navigate with certain displays in TLC space, such as the VB viewer and 3D Airway Tree Renderer. The airway tree's intrinsic geometry remains unchanged regardless of the breathing state and that the airway tree is known to be torsionally rigid. Thus, as the real and virtual bronchoscopes move through their respective spaces with consistent roll angles and intra-branch rotations, then the relative airway bifurcations (and their orientations) observed by the two bronchoscopes will be the same, even though their shapes and sizes can differ somewhat. By this idea, navigation with the “ideal” information could help during the live procedure.

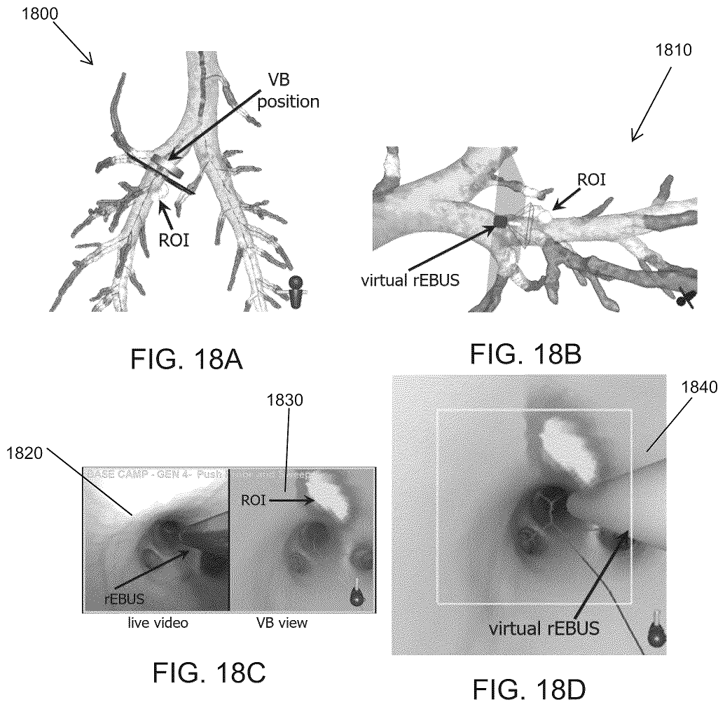

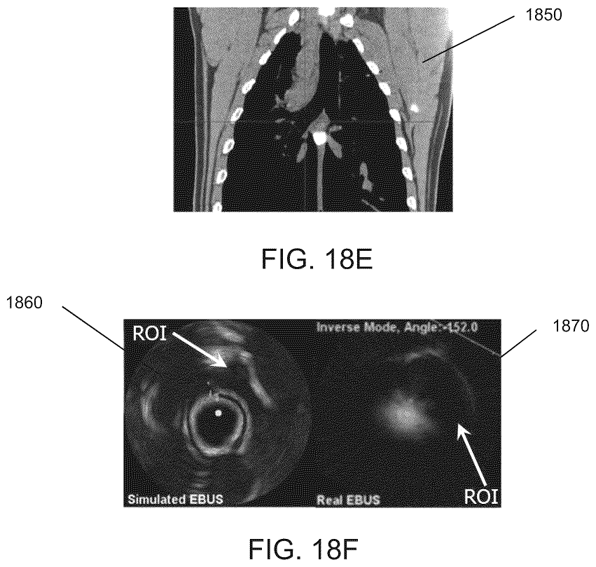

Additionally, supplemental viewers geared toward confirmation/localization supply useful guidance information for confirming an ROI and for deploying a supplemental device, such as an rEBUS probe. For example, FIGS. 18A-18F illustrate several concepts relevant to confirmation and/or localization including: a) a local peripheral renderer that depicts the bronchoscope tip and rEBUS scan plane in relation to the ROI; b) a CT-based simulated rEBUS view at the final destination; and c) a depth-of-sample view of the ROI in the VB view indicating the quality of the viewing location for the ROI to highlight the maximum ROI viewing location. FIGS. 18A-18F also illustrate All views depicted in FIGS. 18A-18F are synchronized at a final destination. Generally, FIGS. 18A-18F illustrate a final “Push probe and sweep” cue at this optimal pose. The location of the real rEBUS probe is clearly synchronized to a virtual rEBUS probe at the proper airway site—leading to an immediate correct confirmation of the ROI with the real rEBUS probe. Such supplemental viewers could obviate the need for local cone beam CT scanning (and the extra radiation such a scan requires), as they conceivably provide the same information and also help guide placement of the rEBUS probe. This completes the procedure.

More specifically, FIG. 18A depicts a 3D airway tree rendering 1800 depicting a model of the bronchoscope tip. The 3D airway tree can be color coded, or otherwise annotated, to reflect if the bronchoscope is able to fit within the airway. For example, green can indicate the bronchoscope will fit, yellow can indicate the bronchoscope will marginally fit, and/or red can indicate the airway is too narrow for the bronchoscope to fit, or otherwise pass through without damaging the airway, for example. FIG. 18B depicts a local peripheral renderer 1810 depicting a rendering of the airway tree, bronchoscope-tip model, and ROI at the final destination. The tip model also shows the rEBUS scan plane. FIG. 18C depicts a live bronchoscope video 1820 depicting real rEBUS probe tip and corresponding VB view 1830 at the final destination with guidance cue. FIG. 18D depicts a panorama renderer 1840 depicting an expanded field-of-view VB view at the final destination, with virtual rEBUS probe tip also shown. FIGS. 18C and 18D depict a depth-of-sample view of the ROI. FIG. 18E depicts a standard 2D axial-plane CT section 1850, and FIG. 18F depicts a CT-based simulated rEBUS view 1860 at the final destination and a live rEBUS view 1870 after engaging the probe.

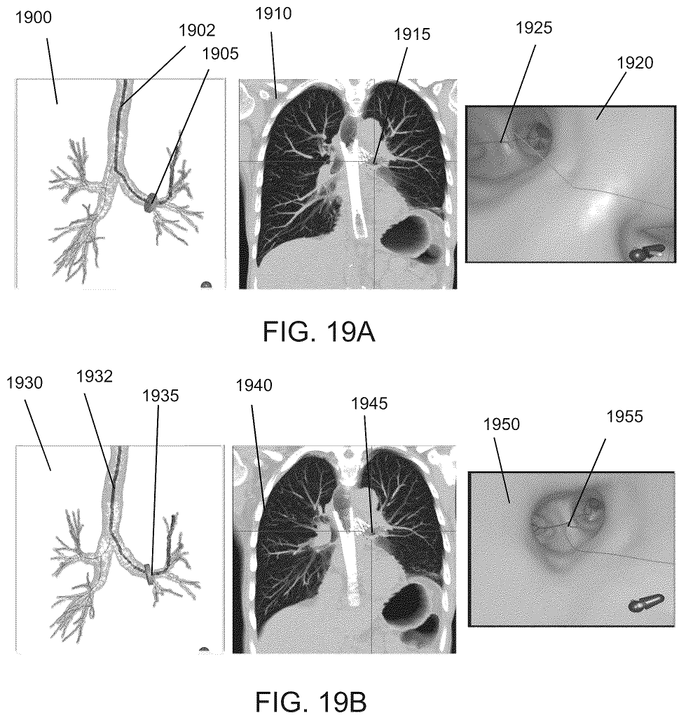

FIGS. 19A-22B illustrate various concepts discussed herein and point out the impact of CTBD during navigation. The example follows the navigation toward the periphery of the left upper lobe (LUL) for a human subject.

FIGS. 19A and 19B show guidance system views that could be observed while navigating in TLC space or FRC space at the endpoint of the generation-2 branch (left main bronchus). While the VB views, for example, aren't identical, it is clear that both paths are essentially at the same physical location within the airway tree. The differences between the pose depicted in FIG. 19A and the pose depicted in FIG. 19B at this view site essentially entail a rotation difference and an indication that the upcoming child airways will be getting smaller in the FRC view more quickly than in the TLC view. More specifically, FIG. 19A depicts a result in TLC space. A 3D rendered airway tree 1900 highlights an active guidance route 1902 and current view site 1905. A coronal front-to-back CT slab rendering 1910 is also shown depicting the current view site by cross hairs 1915. A VB view 1920 depicts a guidance route 1925. FIG. 19B depicts a result in FRC space. Similarly to FIG. 19A, a 3D rendered airway tree 1930 highlights an active guidance route 1932 and current view site 1935 in FIG. 19B. Likewise, a coronal front-to-back CT slab rendering 1940 is shown depicting the current view site by cross hairs 1945 and a VB view 1950 depicts a guidance route 1955.

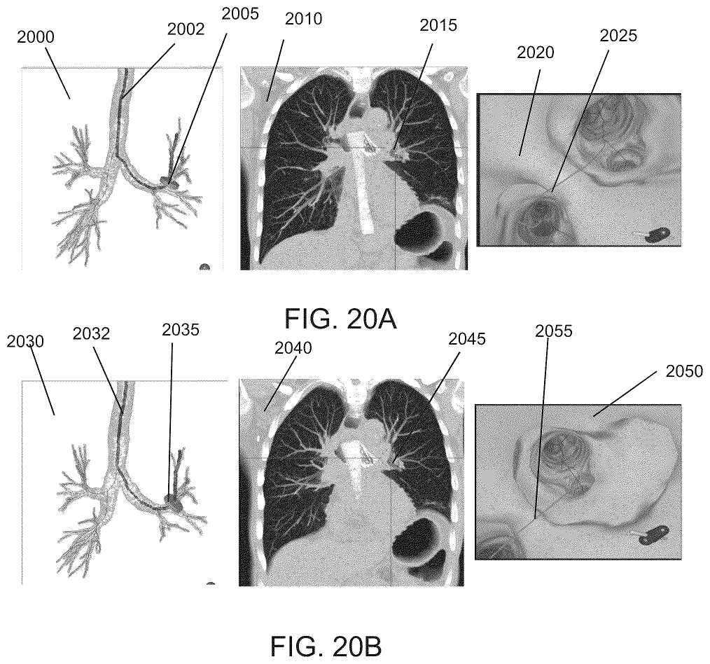

Similar to FIGS. 19A and 19B, the same observations can be observed at the endpoint of the generation-4 branch, as shown in FIGS. 20A and 20B. Making comparisons at the structurally/topologically same locations (the bifurcation point at the end of branch 4) in TLC and FRC reveals clear synchrony in the navigation. More specifically, FIG. 20A depicts a result in TLC space. A 3D rendered airway tree 2000 highlights an active guidance route 2002 and current view site 2005. A coronal front-to-back CT slab rendering 2010 is also shown depicting the current view site by cross hairs 2015. A VB view 2020 depicts a guidance route 2025. FIG. 20B depicts a result in FRC space. Similarly to FIG. 20A, a 3D rendered airway tree 2030 highlights an active guidance route 2032 and current view site 2035 in FIG. 20B. Likewise, a coronal front-to-back CT slab rendering 2040 is shown depicting the current view site by cross hairs 2045 and a VB view 2050 depicts a guidance route 2055.

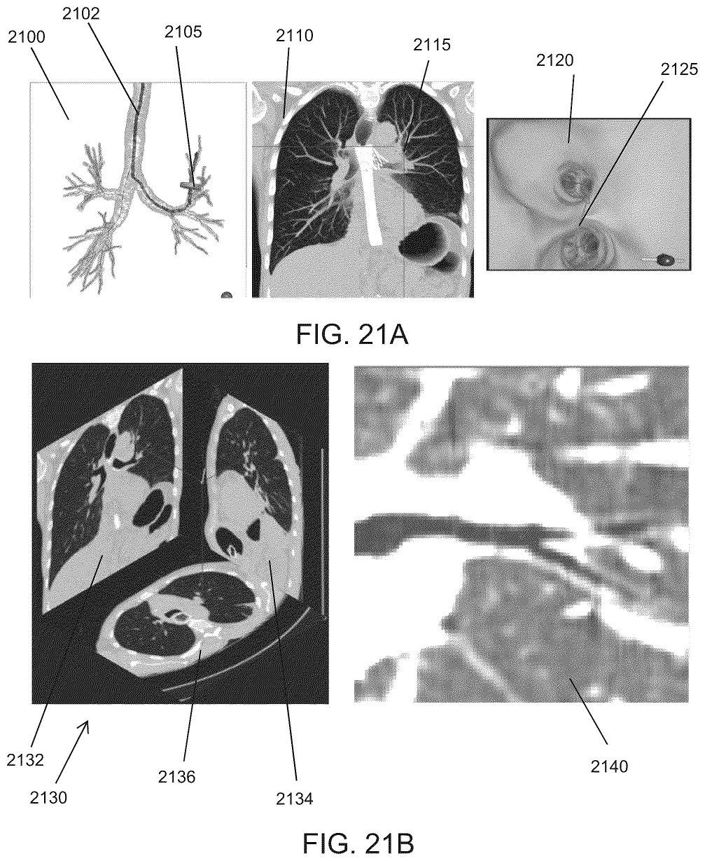

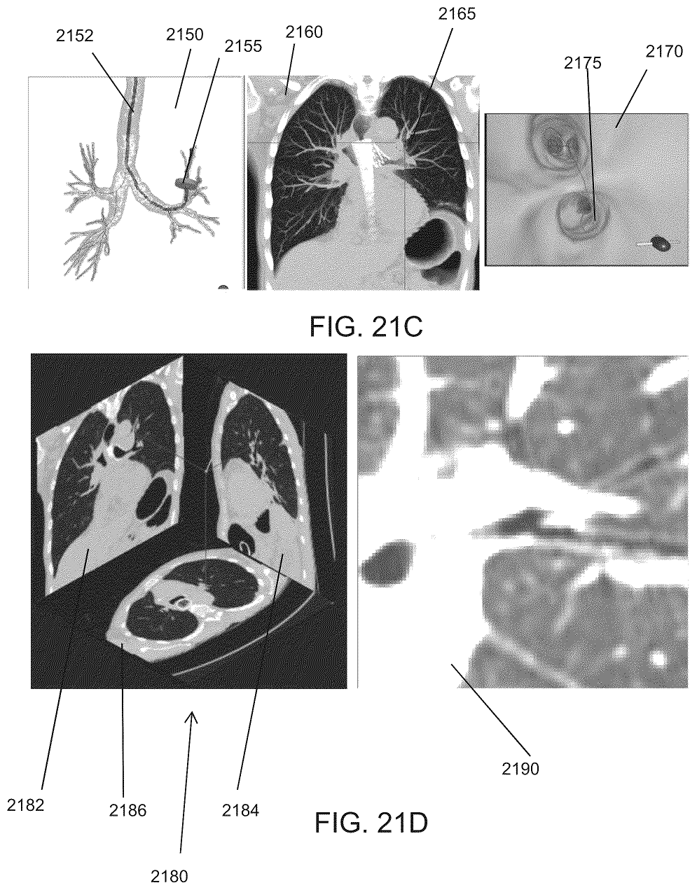

FIGS. 21A-21D provide a more detailed view of this clear synchrony between the two spaces when reaching the end of the generation-6 branch. The 2D local horizontal CT cross-section views at these locations in TLC and FRC add reassurance that both routes are in synchrony. This location could also serve as an example of when the guidance can switch from navigation with the primary guidance route rₚ in TLC space to the final guidance route r_{f} in FRC space; i.e., the end point signifies localization pose v_{L}∈rₚ, and, per (19), b₁∈rₚ corresponds to \hat{b}₁∈r_{f}, b₂∈rₚ corresponds to \hat{b}₂∈r_{f}, . . . b₆∈rₚ corresponds to \hat{b}₆∈r_{f}. FIGS. 21A-21D give confidences that the guidance system is still in the same airway when switching to FRC space for final confirmation/localization.

FIGS. 21A-21D show guidance system views that could be observed while navigating in TLC space or FRC space at the endpoint of the generation-6 branch. FIG. 21A depicts a result in TLC space. A 3D rendered airway tree 2100 highlights an active guidance route 2102 and current view site 2105. A coronal front-to-back CT slab rendering 2110 is also shown depicting the current view site by cross hairs 2115. A VB view 2120 depicts a guidance route 2125. FIG. 21B depicts a “cube” view 2130 of the 2D coronal 2132, sagittal 2134, and axial 2136 plane CT sections at the current view site. FIG. 21B further depicts a local horizontal 2D CT cross-section 2140 centered about the current view site. FIG. 21C depicts a result in FRC space. Similarly to FIG. 21A, a 3D rendered airway tree 2150 highlights an active guidance route 2152 and current view site 2155 in FIG. 21C. Likewise, a coronal front-to-back CT slab rendering 2160 is shown depicting the current view site by cross hairs 2165 and a VB view 2170 depicts a guidance route 2175. Similarly to FIG. 21B, FIG. 21D depicts a “cube” view 2180 of the 2D coronal 2182, sagittal 2184, and axial 2186 plane CT sections at the current view site. FIG. 21D further depicts a local horizontal 2D CT cross-section 2190 centered about the current view site.

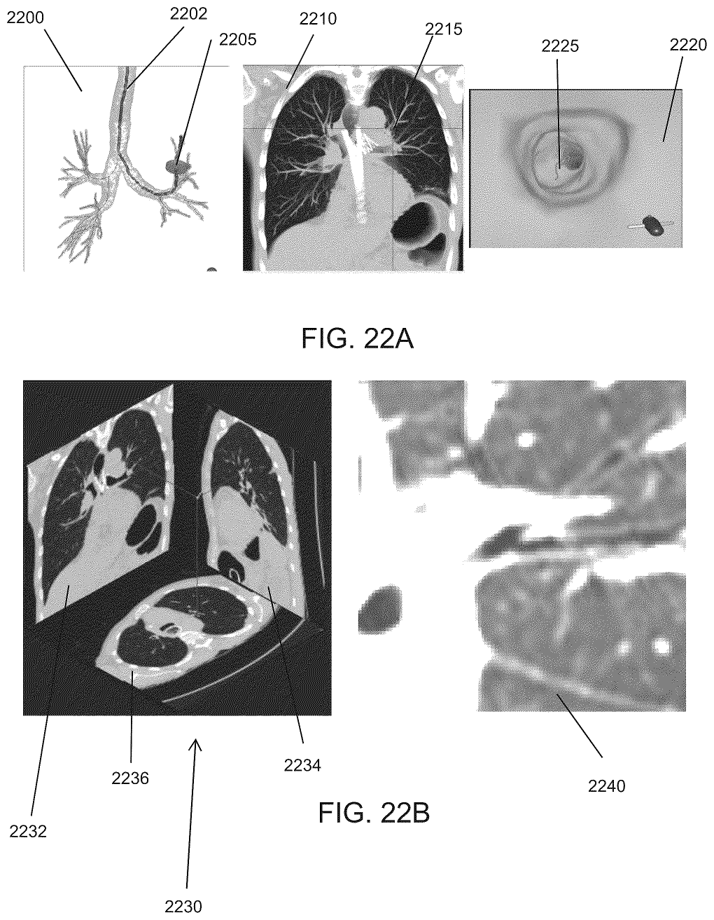

Lastly, FIGS. 22A and 22B depict a result if CTBD is disregarded. For this example, the system traveled a total of 143 view sites in TLC space and 129 view sites in FRC space when navigating from the end point of generation 2 to the end point of generation 6. The view sites are approximately equally spaced 0.6 mm apart. It is assumed the FRC-based space corresponds to the reality during the live procedure. If distance only is used to depict where the system is during navigation and navigation is done only in TLC space, then the guidance system's display will show the results of FIGS. 21A and 21B at the end of the generation-6 airway—14 more view sites will have been navigated than necessary in FRC space (the distance traveled is logged by the scope insertion depth dᵢₙ). Unfortunately, in reality, in FRC space, the system would in fact be at the FRC location shown in FIGS. 22A and 22B, which is part way into the generation 7 airway, approximately 8-9 mm further down stream. The present disclosure, by virtue of the way it maps routes from TLC space to FRC space, would not present this issue.

More specifically, FIG. 22A depicts a result in FRC space. A 3D rendered airway tree 2200 highlights an active guidance route 2202 and a current view site 2205. A coronal front-to-back CT slab rendering 2210 is shown depicting the current view site by cross hairs 2215 and a VB view 2220 depicts a guidance route 2225. FIG. 22B depicts a “cube” view 2230 of the 2D coronal 2232, sagittal 2234, and axial 2236 plane CT sections at the current view site. FIG. 22B further depicts a local horizontal 2D CT cross-section 2240 centered about the current view site.

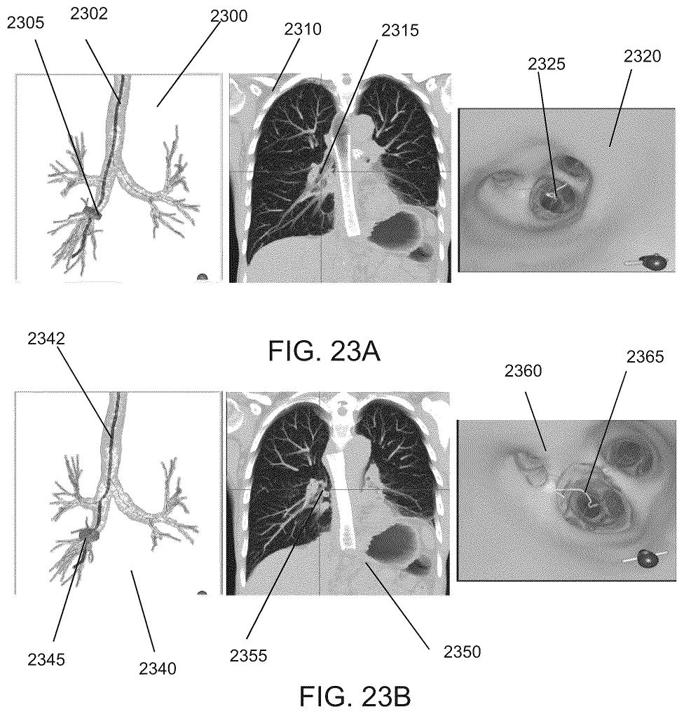

As a second example, FIGS. 23A and 23B illustrate what happens when a clinician starts at the end of the generation-2 airway for a guidance route traveling to the periphery of the right lower lobe. Navigation is then performed in both the TLC- and FRC-based spaces for 150 additional view sites, with no regard to the branch endpoints being passed. In principle, the system views for both cases travel the same distance. Yet, the views in FIGS. 23A and 23B clearly differ. In fact, for the TLC view shown in FIG. 23A, the system is situated at the beginning of the generation-5 airway branch, whereas the FRC view shown in FIG. 23B is much further down the generation-5 branch. It is clear that if the TLC space is used exclusively for navigation, with no regard for the topology of the airway tree, then error arising from CTBD will accumulate.

More specifically, FIG. 23A depicts a result in TLC space. A 3D rendered airway tree 2300 highlights an active guidance route 2302 and a current view site 2305. A coronal front-to-back CT slab rendering 2310 is shown depicting the current view site by cross hairs 2315 and a VB view 2320 depicts a guidance route 2325. FIG. 23B depicts a result in FRC space. A 3D rendered airway tree 2340 highlights an active guidance route 2342 and a current view site 2345. A coronal front-to-back CT slab rendering 2350 is shown depicting the current view site by cross hairs 2355 and a VB view 2360 depicts a guidance route 2365.

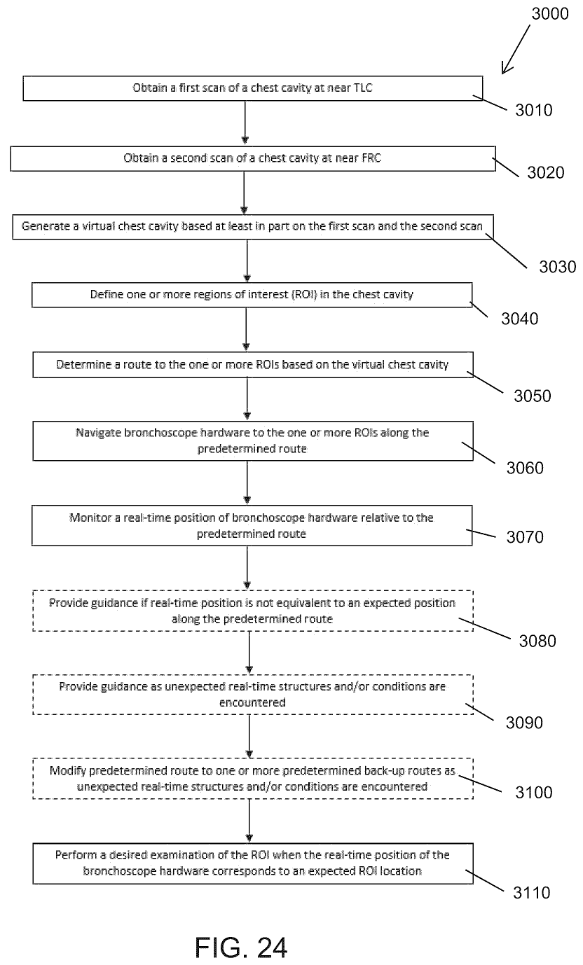

FIG. 24 depicts a flow chart of an exemplary method according to one or more aspects described herein. The method 3000 begins at blocks 3010 and 3020 by obtaining a first scan of a chest cavity at near TLC and a second scan of a chest cavity at near FRC, respectively. The method 3000 continues at block 3030 by generating a virtual chest cavity based at least in part on the first scan and the second scan. During the generation of the virtual chest cavity, a 3D transformation indicating how each point in the first (TLC) CT scan maps to a corresponding point in the second (FRC) CT scan is computed. The method 3000 continues at block 3040 by defining one or more regions of interest (ROI) in the chest cavity. Such ROIs can be defined in the virtual chest cavity, on the first scan, and/or on the second scan. The method 3000 continues at block 3050 by determining a route to the one or more ROIs based on the virtual chest cavity. At block 3060, bronchoscope hardware is directed, or otherwise navigated, to the one or more ROIs along the predetermined route. During the live procedure, as shown in block 3070, a real-time position of the bronchoscope hardware is monitored relative to an expected position along the predetermined route. Optionally, as shown at block 3080, the system can provide a guidance to the user if the real-time position of the bronchoscope hardware is not equivalent, or otherwise does not correspond, to the expected position along the predetermined route. The guidance can be communicated to the user as visual feedback, audio feedback, and/or haptic feedback, for example. Optionally, as shown at block 3090, the system can provide a guidance to the user as an unexpected structure and/or condition is encountered during the live procedure. Such guidance can be communicated to the user as visual feedback, audio feedback, and/or haptic feedback, for example. At block 3100, the method 3000 can optionally include modifying the predetermined route to one or more predetermined back-up routes as the unexpected structure and/or condition is encountered during the live procedure. In various instances, the user has the ability to override any route suggested by the method 3000. The method 3000 concludes at block 3110 by performing a desired examination of the ROI when the real-time position of the bronchoscope hardware corresponds to an expected ROI location.

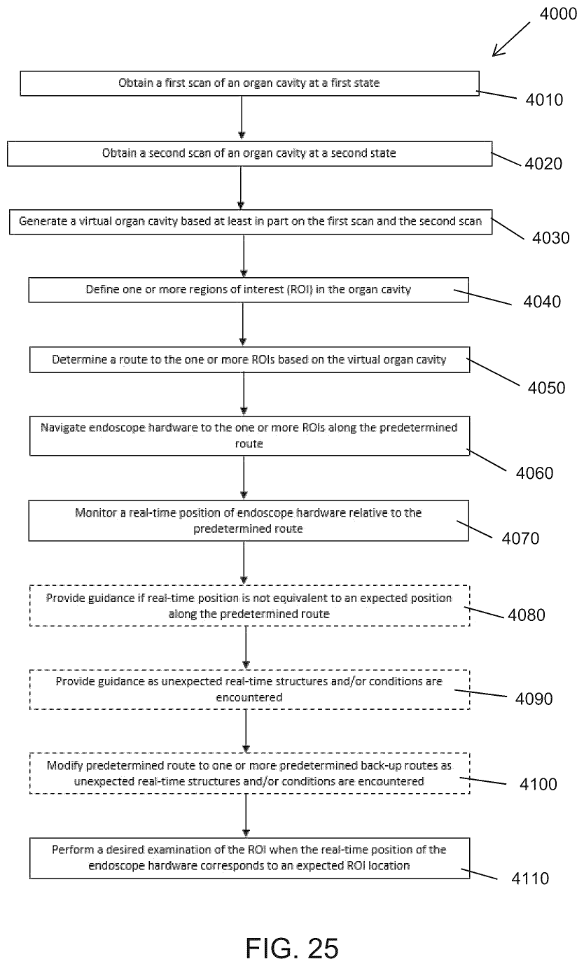

FIG. 25 depicts a flow chart of an exemplary method according to one or more aspects described herein. The method 4000 begins at blocks 4010 and 4020 by obtaining a first scan of an organ cavity in a first state and a second scan of the organ cavity in a second state, respectively. The first state is different from the second state. The method 4000 continues at block 4030 by generating a virtual organ cavity based at least in part on the first scan and the second scan. During the generation of the virtual organ cavity, a 3D transformation indicating how each point in the first scan maps to a corresponding point in the second scan is computed. The method 4000 continues at block 4040 by defining one or more regions of interest (ROI) in the organ cavity. Such ROIs can be defined in the virtual organ cavity, on the first scan, and/or on the second scan. The method 4000 continues at block 4050 by determining a route to the one or more ROIs based on the virtual organ cavity. At block 4060, endoscope hardware is navigated to the one or more ROIs along the predetermined route. During the live procedure, as shown in block 4070, a real-time position of the endoscope hardware is monitored relative to an expected position along the predetermined route. Optionally, as shown at block 4080, the system can provide a guidance to the user if the real-time position of the endoscope hardware is not equivalent, or otherwise does not correspond, to the expected position along the predetermined route. The guidance can be communicated to the user as visual feedback, audio feedback, and/or haptic feedback, for example. Optionally, as shown at block 4090, the system can provide a guidance to the user as an unexpected structure and/or condition is encountered during the live procedure. Such guidance can be communicated to the user as visual feedback, audio feedback, and/or haptic feedback, for example. At block 4100, the method 4000 can optionally include modifying the predetermined route to one or more predetermined back-up routes as the unexpected structure and/or condition is encountered during the live procedure. In various instances, the user has the ability to override any route suggested by the method 4000. The method 4000 concludes at block 4110 by performing a desired examination of the ROI when the real-time position of the endoscope hardware corresponds to an expected ROI location.

Notably, the planning methodologies detailed herein are also applicable to robotics-assisted bronchoscopy systems. Robotics assisted bronchoscopy systems have special hardware to provide automated movement of the bronchoscope during navigation. The robotics system can glean navigation information either from an electromagnetic sensor-based synchronization mechanism that ascertains scope tip location or a shape-sensing sensor driven by a fiber-optic shape sensor that measures the complete shape of the device during the live procedure. For robotics-assisted bronchoscopy systems, initial set-up in the operating room entails essentially the same steps, as described above, except that a robotics-assisted bronchoscopy system drawing upon shape sensing is now used during the live procedure. The procedure plan is loaded and video output from the bronchoscope is made available. In addition, the guidance system's display is initialized for ROI R, as performed above.

With the active guidance route initialized to the preplanned final guidance route rₚ, the clinician uses the robotic system to initialize the bronchoscope near the main carina. Also, the scope insertion depth is initialized to dᵢₙ=0 mm, and the corresponding remaining distance to the ROI d_{ROI} is initialized based on route rₚ. Live guidance again follows a three-stage protocol toward R: (1) navigating toward the ROI; (2) optionally switching to a back-up guidance route; and (3) finally confirming and/or localizing the ROI. As before, linked TLC-space and FRC-space versions of all graphical viewers are available by toggling the guidance space.

Navigation proceeds with the robotic system following the active guidance route and associated guidance cues to maneuver the bronchoscope. As before, based on the display information, the clinician can redirect the robotic system to any available back-up guidance route rⁿ, as deemed necessary. During the procedure, dᵢₙ and d_{ROI} can receive constant, or otherwise routine, updates. In addition, d_{ROI} stays synchronized to the currently active guidance route, regardless of which route is active. The robotic system maintains the current pose information for the bronchoscope tip, as it is maneuvered; i.e., the tip's 3D position, Euler angles, and/or up vector are constantly, or otherwise routinely, updated, based on the shape sensing technology. Notably, because dᵢₙ is an absolute measure independent of the guidance route and navigation space (TLC-based or FRC-based), dᵢₙ does not change when the active guidance changes.

When the guidance system reaches the active guidance route's localization pose V_{L} (again, typically 2 to 4 cm from the final destination), the system switches the active guidance route to the complementary final guidance route, be it r_{f} or r_{f}ⁿ, situated in FRD-based CT virtual space. This causes d_{ROI} to be updated to FRC-based virtual space, while d_{ID} remains unchanged as the bronchoscope has not moved.

Because of CTBD between the TLC and FRC spaces, it is important to note that when the route switch is made, the displayed live bronchoscope video and the corresponding displayed VB view can differ significantly. To adjust for this disparity, two operations are performed. First, the view site \hat{v}_{β,j} on the active guidance route situated d_{ID} mm from the beginning of the route is set to be the guidance system's current view site, where \hat{v}_{β,j} is the β^{th} view site of branch \hat{b}ⱼ of the active guidance route. Notably, d_{ID} corresponds to the total distance navigated thus far by the bronchoscope toward ROI R. This places the guidance system's virtual bronchoscope, now situated in FRC-based virtual space at a pose Θ_{β,j}, closer to the current pose Θ of the real bronchoscope situated in real chest space. To reduce the remaining disparity between the virtual-space pose Θ_{β,j} and real-space pose Θ, an image-based registration of VB view I_{CT}^{Θ}^{_{β,j}} to the current live video bronchoscope view I_{V}^{Θ} is performed. The view site on the active guidance route that is closest to the derived optimal virtual-space pose Θₒ is now ascertained, with the direction information from Θₒ also retained. This completes the synchronization of the two spaces. The remaining bronchoscope maneuvers are now performed using the robotics system until the bronchoscope reaches the preplanned optimal destination pose. As before, TLC-based information is available and synchronized to the current active guidance route.

As described above, derivation and/or utilization of back-up routes is an optional attribute of the disclosed methods.

### EXAMPLES

Example 1—A method for planning and guiding an endoscopic procedure taking place in a hollow tubular structure inside a subject's multi-organ cavity that requires an endoscope, the method including the steps of: receiving two 3D pre-operative imaging scans of the subject depicting the multi-organ cavity, with the first pre-operative imaging scan depicting the multi-organ cavity at a different state from the second pre-operative imaging scan; computing an anatomical model for each 3D pre-operative imaging scan, with each model including the definition and centerlines of the hollow tubular structure and the definitions of the organs inside the multi-organ cavity; identifying and defining a region of interest (ROI) inside the multi-organ cavity in the first 3D pre-operative imaging scan; deriving a 3D transformation that maps each point in the first 3D preoperative imaging scan to a corresponding point in the second 3D pre-operative imaging scan, based on the two 3D pre-operative imaging scans and anatomical models; and deriving a procedure plan based on the first 3D preoperative imaging scan and its anatomical model, the ROI, and endoscope device characteristics, the procedure plan including an initial guidance route inside the hollow tubular structure leading to a localization pose and then terminating at an initial destination pose near the ROI and guidance cues indicating how to maneuver the endoscope along the initial guidance route toward the initial destination pose. The method further includes mapping one or more points of the initial guidance route into corresponding points in the second 3D pre-operative imaging scan, based on the 3D transformation, thereby providing a preliminary final guidance route based on the second 3D pre-operative imaging scan; deriving a final guidance route inside the hollow tubular structure leading to a final destination pose near the ROI and guidance cues indicating how to maneuver the endoscope along the final guidance route toward the final destination pose, based on the second 3D pre-operative imaging scan and its anatomical model, the ROI, endoscope device characteristics, and the preliminary final guidance route; and performing a live two-stage guidance protocol. The live two-stage guidance protocol includes providing guidance information which enables the endoscope operator to navigate, or otherwise direct, the endoscope to a preplanned destination pose near the ROI, which includes the steps of updating the active guidance route to the preplanned initial guidance route, providing simulated virtual video views of the hollow tubular structure along the active guidance route from a virtual endoscope defined within a 3D virtual space and constructed based on the first 3D pre-operative imaging scan and its anatomical model and the ROI and also providing the preplanned guidance cues, which enable the endoscope operator to navigate the endoscope toward the active guidance route's preplanned destination pose near the ROI, initializing the endoscope's position within the subject's hollow tubular structure and distance to the preplanned destination pose, based on the simulated virtual video views of the hollow tubular structure and the active guidance route, providing updates of the distance to the preplanned destination pose as the virtual endoscope advances along the active guidance route toward the preplanned destination pose, and updating the endoscope's position within the subject's hollow tubular structure, based on 1) the simulated virtual video views of the hollow tubular structure, 2) the active guidance route and 3) the preplanned guidance cues. The live two-stage guidance protocol further includes upon reaching the active guidance route's preplanned localization pose, providing guidance information which enables the endoscope operator to localize the endoscope at the preplanned final guidance route's destination pose near the ROI, which includes the steps of updating the active guidance route the final guidance route, updating the endoscope's position within the subject's hollow tubular structure and distance to the preplanned destination pose, based on the simulated virtual video views of the hollow tubular structure and the active guidance route, providing simulated virtual video views of the hollow tubular structure along the active guidance route from a virtual endoscope defined within a 3D virtual space and constructed based on the second 3D pre-operative imaging scan and its anatomical model and the ROI and also providing the preplanned guidance cues which enable the endoscope operator to maneuver the endoscope toward the active guidance route's preplanned destination pose, providing updates of the distance to the preplanned destination pose as the virtual endoscope advances along the active guidance route toward the preplanned destination pose, updating the endoscope's position within the subject's hollow tubular structure, based on the simulated virtual video views of the hollow tubular structure, the active guidance route and the preplanned guidance cues, and upon reaching the preplanned destination pose, providing guidance information which enables the endoscope operator to localize the endoscope at the preplanned destination pose. The 3D positions of the virtual endoscope and real endoscope are synchronized at a final destination pose for examining the ROI.

Example 2—The method of Example 1, wherein the method receives endoscope tip location and insertion depth information from a robotics assisted endoscopy system during the live two-stage guidance protocol for the purpose of updating the endoscope's position within the subject's hollow tubular structure.

Example 3—A system for pre-operative planning and live guidance of an endoscopic procedure taking place in a hollow tubular structure inside a multi-organ cavity that requires an endoscope, drawing on the methods of any of Examples 1 and 2.

Example 4—A method for planning and guiding an endoscopic procedure taking place in a hollow tubular structure inside a subject's multi-organ cavity that requires an endoscope is disclosed. The method includes receiving two 3D pre-operative imaging scans of the subject depicting the multi-organ cavity, with the first pre-operative imaging scan depicting the multi-organ cavity at a different state from the second pre-operative imaging scan; computing an anatomical model for each 3D pre-operative imaging scan, with each model consisting of 1) the definition and centerlines of the hollow tubular structure, where the centerlines consist of a series of connected ordered branches, and 2) the definitions of the organs inside the multi-organ cavity; identifying and defining a region of interest (ROI) inside the multi-organ cavity in the first 3D pre-operative imaging scan; deriving a 3D transformation that maps each point in the first 3D preoperative imaging scan to a corresponding point in the second 3D pre-operative imaging scan, based on the two 3D pre-operative imaging scans and anatomical models; and deriving a procedure plan based on 1) the first 3D preoperative imaging scan and its anatomical model, 2) the ROI and 3) endoscope device characteristics, the procedure plan including 1) an primary guidance route, which consists of a series of N≥0 connected ordered branches concatenated with a termination segment, inside the hollow tubular structure leading to a localization pose and then terminating at a destination pose near the ROI and 2) guidance cues indicating how to maneuver the endoscope along the primary guidance route toward the destination pose. The method further includes deriving a series of back-up procedure plans, which includes for each n less than or equal to N starting at n=1, constructing a partial guidance route, if possible, defined by the first n branches constituting the primary guidance route and for each n less than or equal to N starting at n=1, deriving a back-up procedure plan, if possible, based on 1) the first 3D preoperative imaging scan and its anatomical model, 2) the ROI and 3) endoscope device characteristics, the back-up procedure plan including 1) a back-up guidance route inside the hollow tubular structure, where the back-up guidance route consists of the n-branch partial guidance route concatenated with other connected ordered branches and a terminating segment leading to a back-up localization pose and then terminating at a back-up destination pose near the ROI, and 2) guidance cues indicating how to maneuver the endoscope along the back-up guidance route toward the back-up destination pose. The method further includes mapping one or more points of the primary guidance route into corresponding points in the second 3D pre-operative imaging scan, based on the 3D transformation, thereby providing a preliminary final guidance route based on the second 3D pre-operative imaging scan, mapping one or more points in each of the back-up guidance routes into corresponding points in the second 3D pre-operative imaging scan, based on the 3D transformation, thereby providing a set of preliminary back-up guidance routes based on the second 3D pre-operative imaging scan, deriving a final guidance route inside the hollow tubular structure leading to a final destination pose near the ROI, and guidance cues indicating how to maneuver the endoscope along final guidance route toward the final destination pose based on the second 3D pre-operative imaging scan and its anatomical model, the ROI, endoscope device characteristics, and the preliminary final guidance route; and deriving a set of final back-up guidance routes inside the hollow tubular structure, each route leading to an associated final back-up destination pose near the ROI, and guidance cues indicating how to maneuver the endoscope along each final back-up guidance route toward its respective final back-up destination pose based on 1) the second 3D pre-operative imaging scan and its anatomical model, 2) the ROI, 3) endoscope device characteristics and 4) the set of preliminary back-up guidance routes. The method further includes performing a live three-stage guidance protocol including the steps of providing guidance information which enables the endoscope operator to navigate the endoscope to a preplanned destination pose near the ROI which includes the steps of updating the active guidance route to the preplanned primary guidance route, providing simulated virtual video views of the hollow tubular structure along the active guidance route from a virtual endoscope defined within a 3D virtual space and constructed based on 1) the first 3D pre-operative imaging scan and its anatomical model and 2) the ROI and also providing the preplanned guidance cues, which enable the endoscope operator to maneuver the endoscope toward the active guidance route's preplanned destination pose near the ROI, initializing the endoscope's position within the subject's hollow tubular structure and distance to the preplanned destination pose, based on the simulated virtual video views of the hollow tubular structure and the active guidance route, providing updates of the distance to the preplanned destination pose as the virtual endoscope advances along the active guidance route toward the preplanned destination pose, and updating the endoscope's position within the subject's hollow tubular structure, based on 1) the simulated virtual video views of the hollow tubular structure, 2) the active guidance route and 3) the preplanned guidance cues. The live three-stage guidance protocol further includes for each n less than or equal to N starting at n=1, if possible, providing guidance information which enables the endoscope operator to switch the active guidance route to a preplanned back-up guidance route and continue navigating the endoscope to the preplanned back-up guidance route's back-up destination pose near the ROI, which includes the steps of upon reaching the end of branch n of the active guidance route, signaling the endoscope operator that a preplanned back-up guidance route is available for reaching the ROI, updating the active guidance route to the preplanned back-up guidance route, updating the endoscope's position within the subject's hollow tubular structure and distance to the preplanned destination pose, based on the simulated virtual video views of the hollow tubular structure and the active guidance route, providing simulated virtual video views of the hollow tubular structure along the active guidance route from a virtual endoscope defined within a 3D virtual space and constructed based on 1) the first 3D pre-operative imaging scan and its anatomical model and 2) the ROI and also providing the corresponding preplanned guidance cues, which enable the endoscope operator to maneuver the endoscope toward the active guidance route's preplanned destination pose, providing updates of the distance to the preplanned destination pose as the virtual endoscope advances along the active guidance route toward the preplanned destination pose, and updating the endoscope's position within the subject's hollow tubular structure, based on 1) the simulated virtual video views of the hollow tubular structure, 2) the active guidance route and 3) the preplanned guidance cues. The three-stage guidance protocol further includes upon reaching the active guidance route's localization pose, providing guidance information which enables the endoscope operator to localize the endoscope at the final destination pose near the ROI of the active guidance route's associated final guidance route (primary or back-up), which includes the steps of updating the active guidance route to the active guidance route's associated final guidance route, updating the endoscope's position within the subject's hollow tubular structure and distance to the preplanned destination pose, based on the simulated virtual video views of the hollow tubular structure and the active guidance route, providing simulated virtual video views of the hollow tubular structure along the active guidance route from a virtual endoscope defined within a 3D virtual space and constructed based on 1) the second 3D pre-operative imaging scan and its anatomical model and 2) the ROI and also providing the preplanned guidance cues which enable the endoscope operator to maneuver the endoscope toward the active guidance route's preplanned destination pose, updating the endoscope's position within the subject's hollow tubular structure and distance to the preplanned destination pose, based on the active guidance route, providing updates of the distance to the preplanned destination pose as the virtual endoscope advances along the active guidance route toward the preplanned destination pose, and upon reaching the preplanned destination pose, providing guidance information which enables the endoscope operator to localize the endoscope at the preplanned destination pose near the ROI. The 3D positions of the virtual endoscope and real endoscope are synchronized at a final destination pose for examining the ROI.

Example 5—The method of Example 4, wherein the method receives endoscope tip location and insertion depth information from a robotics assisted endoscopy system during the live three-stage guidance protocol for the purpose of updating the endoscope's position within the subject's hollow tubular structure.

Example 6—A system for pre-operative planning and live guidance of an endoscopic procedure taking place in a hollow tubular structure inside a multi-organ cavity that requires an endoscope, drawing on the methods of any of Examples 4 and 5.

Example 7—A method for planning and guiding an endoscopic procedure is disclosed. The method includes obtaining a first scan of a chest cavity at a near total lung capacity, obtaining a second scan of the chest cavity at a near functional residual capacity, generating a virtual chest cavity based at least in part on the first scan and the second scan, defining one or more regions of interest (ROI) in the virtual chest cavity, and determining a route to the one or more ROIs based on the virtual chest cavity. The method further includes directing endoscope hardware through an airway to the one or more ROIs along the predetermined route, monitoring, by a processor, a real-time position of the endoscope hardware relative to an expected position along the predetermined route, and performing an examination of the one or more ROIs when the real-time position of the endoscope hardware corresponds to an expected ROI location.

Example 8—The method of Example 7, further including providing a guidance to a user when the real-time position does not correspond to the expected position along the predetermined route.

Example 9—The method of any of Examples 7-8, further including providing a guidance to a user as one or more unexpected structures are encountered along the predetermined route.

Example 10—The method of Example 9, further including modifying the predetermined route to one or more predetermined back-up routes as the one or more unexpected structures are encountered along the predetermined route.

Example 11—The method of any of Examples 7-10, further including providing a guidance to a user as one or more unexpected conditions are encountered along the predetermined route.

Example 12—The method of Example 11, further including modifying the predetermined route to one or more predetermined back-up routes as the one or more unexpected conditions are encountered along the predetermined route.

Example 13—The method of Example 12, further including overriding the guidance and continuing to direct the endoscope hardware to the one or more ROIs along the predetermined route.

Example 14—The method of any of Examples 7-13, wherein the step of generating a virtual chest cavity includes computing a 3D transformation indicating how a first point in the first scan maps to a corresponding point in the second scan.

Example 15—The method of any of Examples 7-14, further including determining one or more back-up routes prior to the step of directing the endoscope hardware through the airway.

Example 16—A method of planning and guiding an endoscopic procedure is disclosed. The method includes obtaining a first scan of an organ cavity at a first state, obtaining a second scan of the organ cavity at a second state, wherein the first state is different than the second state, generating a virtual organ cavity based at least in part on the first scan and the second scan, defining one or more regions of interest (ROI) in the virtual organ cavity, and determining a route to the one or more ROIs based on the virtual organ cavity. The method further includes directing endoscope hardware along a pathway to the one or more ROIs along the predetermined route, monitoring, by a processor, a real-time position of the endoscope hardware relative to an expected position along the predetermined route, and performing an examination of the one or more ROIs when the real-time position of the endoscope hardware corresponds to an expected ROI location.

Example 16—The method of Example 15, further including providing a guidance to a user if the real-time position does not correspond to the expected position along the predetermined route.

Example 17—The method of any of Examples 15-16, further including providing a guidance to a user as one or more unexpected structures are encountered along the predetermined route.

Example 18—The method of Example 17, further including modifying the predetermined route to one or more predetermined back-up routes as the one or more unexpected structures are encountered along the predetermined route.

Example 19—The method of any of Examples 15-18, further including providing a guidance to a user as one or more unexpected conditions are encountered along the predetermined route.

Example 20—The method of Example 19, further including modifying the predetermined route to one or more predetermined back-up routes as the one or more unexpected conditions are encountered along the predetermined route.

Example 21—The method of Example 20, further including overriding the guidance and continuing to direct the endoscope hardware to the one or more ROIs along the predetermined route.

Example 22—The method of any of Examples 15-21, wherein the step of generating a virtual organ cavity includes computing a 3D transformation indicating how a first point in the first scan maps to a corresponding point in the second scan.

Example 23—The method of any of Examples 15-22, further including determining one or more back-up routes prior to the step of directing the endoscope hardware along the pathway.

Example 24—A method of planning and guiding an endoscopic procedure is disclosed. The method includes obtaining a first scan of an organ cavity at a first state, obtaining a second scan of the organ cavity at a second state, wherein the first state is different than the second state, computing an anatomical model for each of the first scan and the second scan, defining one or more regions of interest (ROI) in the organ cavity, deriving a 3D transformation that maps each point in the first scan to each corresponding point in the second scan, and deriving a procedure plan including a primary route to the one or more ROIs. The method further includes directing endoscope hardware along a pathway to the one or more ROIs, wherein the pathway extends along the primary route, monitoring, by a processor, a real-time position of the endoscope hardware relative to an expected position along the primary route, and performing a desired examination of the one or more ROIs when the real-time position of the endoscope hardware corresponds to an expected ROI location.

Example 25—The method of Example 24, further including deriving one or more back-up routes prior to the step of directing the endoscope hardware along the pathway.

Example 26—A non-transitory, processor-readable storage medium is disclosed. The non-transitory, processor-readable storage medium comprises one or more programming instructions stored thereon that, when executed, cause a processing device to generate a virtual organ cavity based at least in part on a first scan of an organ cavity at a first state and a second scan of the organ cavity at a second state, wherein the first state is different than the second state, define one or more regions of interest (ROI) in the virtual organ cavity, determine a route to the one or more ROIs based on the virtual organ cavity, and monitor a real-time position of endoscope hardware relative to an expected position along the predetermined route.

Example 27—The storage medium of Example 26, wherein the one or more programming instructions stored thereon, when executed, further cause the processing device to provide a guidance to a user if the real-time position does not correspond to the expected position along the predetermined route.

Example 28—The storage medium of any of Examples 26-27, wherein the one or more programming instructions stored thereon, when executed, further cause the processing device to provide a guidance to a user as one or more unexpected structures are encountered along the predetermined route.

Example 29—The storage medium of Example 28, wherein the one or more programming instructions stored thereon, when executed, further cause the processing device to modify the predetermined route to one or more predetermined back-up routes as the one or more unexpected structures are encountered along the predetermined route.

Example 30—The storage medium of any of Examples 25-29, wherein the one or more programming instructions stored thereon, when executed, further cause the processing device to provide a guidance to a user as one or more unexpected conditions are encountered along the predetermined route.

Example 31—The storage medium of Example 30, wherein the one or more programming instructions stored thereon, when executed, further cause the processing device to modify the predetermined route to one or more predetermined back-up routes as the one or more unexpected conditions are encountered along the predetermined route.

Example 32—The storage medium of Example 31, wherein the one or more programming instructions stored thereon, when executed, further cause the processing device to override the guidance and continuing to direct the endoscope hardware to the one or more ROIs along the predetermined route.

Example 33—A system for planning and guiding an endoscopic procedure is disclosed. The system includes a processing device and a non-transitory, processor-readable storage medium. The storage medium includes one or more programming instructions stored thereon that, when executed, cause the processing device to generate a virtual organ cavity based at least in part on a first scan of an organ cavity at a first state and a second scan of the organ cavity at a second state, wherein the first state is different than the second state, define one or more regions of interest (ROI) in the virtual organ cavity, determine a route to the one or more ROIs based on the virtual organ cavity, and monitor a real-time position of endoscope hardware relative to an expected position along the predetermined route.

Example 34—The system of Example 33, wherein the one or more programming instructions stored thereon, when executed, further cause the processing device to provide a guidance to a user if the real-time position does not correspond to the expected position along the predetermined route.

Example 35—The system of any of Examples 33-34, wherein the one or more programming instructions stored thereon, when executed, further cause the processing device to provide a guidance to a user as one or more unexpected structures are encountered along the predetermined route.

Example 36—The system of Example 35, wherein the one or more programming instructions stored thereon, when executed, further cause the processing device to modify the predetermined route to one or more predetermined back-up routes as the one or more unexpected structures are encountered along the predetermined route.

Example 37—The system of any of Examples 33-36, wherein the one or more programming instructions stored thereon, when executed, further cause the processing device to provide a guidance to a user as one or more unexpected conditions are encountered along the predetermined route.

Example 38—The system of Example 37, wherein the one or more programming instructions stored thereon, when executed, further cause the processing device to modify the predetermined route to one or more predetermined back-up routes as the one or more unexpected conditions are encountered along the predetermined route.

Example 39—The system of Example 38, wherein the one or more programming instructions stored thereon, when executed, further cause the processing device to override the guidance and continuing to direct the endoscope hardware to the one or more ROIs along the predetermined route.

It should now be understood that the present disclosure relates to both systems and/or methods for planning and guiding an endoscopic procedure that assist a clinician in navigating, or otherwise directing, an endoscope through hollow regions situated inside an organ cavity, where the objective of the endoscopic procedure is to examine a preplanned target ROI. Because the organ cavity may change in shape over time, the methods and/or systems compensate for the image-to-body differences that can arise between how the organ cavity appears in imaging scans used to plan the procedure and how the same organ cavity appears during the live endoscopic procedure.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method for planning and guiding an endoscopic procedure, the method comprising:
obtaining a first scan of a chest cavity at a near total lung capacity;
obtaining a second scan of the chest cavity at a near functional residual capacity;
generating a virtual chest cavity based at least in part on the first scan and the second scan;
defining one or more regions of interest (ROI) in the virtual chest cavity;
determining a route to the one or more ROIs based on the virtual chest cavity;
directing endoscope hardware through an airway to the one or more ROIs along the predetermined route;
monitoring, by a processor, a real-time position of the endoscope hardware relative to an expected position along the predetermined route; and
performing an examination of the one or more ROIs when the real-time position of the endoscope hardware corresponds to an expected ROI location.

2. The method of claim 1, further comprising providing a guidance to a user when the real-time position does not correspond to the expected position along the predetermined route.

3. The method of claim 1, further comprising providing a guidance to a user as one or more unexpected structures are encountered along the predetermined route.

4. The method of claim 3, further comprising modifying the predetermined route to one or more predetermined back-up routes as the one or more unexpected structures are encountered along the predetermined route.

5. The method of claim 1, further comprising providing a guidance to a user as one or more unexpected conditions are encountered along the predetermined route.

6. The method of claim 5, further comprising modifying the predetermined route to one or more predetermined back-up routes as the one or more unexpected conditions are encountered along the predetermined route.

7. The method of claim 6, further comprising overriding the guidance and continuing to direct the endoscope hardware to the one or more ROIs along the predetermined route.

8. The method of claim 1, wherein the step of generating a virtual chest cavity comprises computing a 3D transformation indicating how a first point in the first scan maps to a corresponding point in the second scan.

9. The method of claim 1, further comprising determining one or more back-up routes prior to the step of directing the endoscope hardware through the airway.

10. A method of planning and guiding an endoscopic procedure, the method comprising:
obtaining a first scan of an organ cavity at a first state;
obtaining a second scan of the organ cavity at a second state, wherein the first state is different than the second state;
generating a virtual organ cavity based at least in part on the first scan and the second scan;
defining one or more regions of interest (ROI) in the virtual organ cavity;
determining a route to the one or more ROIs based on the virtual organ cavity;
directing endoscope hardware along a pathway to the one or more ROIs along the predetermined route;
monitoring a real-time position of the endoscope hardware relative to an expected position along the predetermined route; and
performing an examination of the one or more ROIs when the real-time position of the endoscope hardware corresponds to an expected ROI location.

11. The method of claim 10, further comprising providing a guidance to a user if the real-time position does not correspond to the expected position along the predetermined route.

12. The method of claim 10, further comprising providing a guidance to a user as one or more unexpected structures are encountered along the predetermined route.

13. The method of claim 12, further comprising modifying the predetermined route to one or more predetermined back-up routes as the one or more unexpected structures are encountered along the predetermined route.

14. The method of claim 10, further comprising providing a guidance to a user as one or more unexpected conditions are encountered along the predetermined route.

15. The method of claim 14, further comprising modifying the predetermined route to one or more predetermined back-up routes as the one or more unexpected conditions are encountered along the predetermined route.

16. The method of claim 15, further comprising overriding the guidance and continuing to direct the endoscope hardware to the one or more ROIs along the predetermined route.

17. The method of claim 10, wherein the step of generating a virtual organ cavity comprises computing a 3D transformation indicating how a first point in the first scan maps to a corresponding point in the second scan.

18. The method of claim 10, further comprising determining one or more back-up routes prior to the step of directing the endoscope hardware along the pathway.

19. A method of planning and guiding an endoscopic procedure, the method comprising:
obtaining a first scan of an organ cavity at a first state;
obtaining a second scan of the organ cavity at a second state, wherein the first state is different than the second state;
computing an anatomical model for each of the first scan and the second scan;
defining one or more regions of interest (ROI) in the organ cavity;
deriving a 3D transformation that maps each point in the first scan to each corresponding point in the second scan;
deriving a procedure plan including a primary route to the one or more ROIs;
directing endoscope hardware along a pathway to the one or more ROIs, wherein the pathway extends along the primary route;
monitoring, by a processor, a real-time position of the endoscope hardware relative to an expected position along the primary route; and
performing a desired examination of the one or more ROIs when the real-time position of the endoscope hardware corresponds to an expected ROI location.

20. The method of claim 19, further comprising deriving one or more back-up routes prior to the step of directing the endoscope hardware along the pathway.

21. A non-transitory, processor-readable storage medium comprising one or more programming instructions stored thereon that, when executed, cause a processing device to:
generate a virtual organ cavity based at least in part on a first scan of an organ cavity at a first state and a second scan of the organ cavity at a second state, wherein the first state is different than the second state;
define one or more regions of interest (ROI) in the virtual organ cavity;
determine a route to the one or more ROIs based on the virtual organ cavity; and
monitor a real-time position of endoscope hardware relative to an expected position along the predetermined route.

22. The storage medium of claim 21, wherein the one or more programming instructions stored thereon, when executed, further cause the processing device to:
provide a guidance to a user if the real-time position does not correspond to the expected position along the predetermined route.

23. The storage medium of claim 21, wherein the one or more programming instructions stored thereon, when executed, further cause the processing device to:
provide a guidance to a user as one or more unexpected structures are encountered along the predetermined route.

24. A system for planning and guiding an endoscopic procedure, the system comprising:
a processing device; and
a non-transitory, processor-readable storage medium comprising one or more programming instructions stored thereon that, when executed, cause the processing device to:
generate a virtual organ cavity based at least in part on a first scan of an organ cavity at a first state and a second scan of the organ cavity at a second state, wherein the first state is different than the second state;
define one or more regions of interest (ROI) in the virtual organ cavity;
determine a route to the one or more ROIs based on the virtual organ cavity; and
monitor a real-time position of endoscope hardware relative to an expected position along the predetermined route.

25. The system of claim 24, wherein the one or more programming instructions stored thereon, when executed, further cause the processing device to:
provide a guidance to a user if the real-time position does not correspond to the expected position along the predetermined route.

26. The system of claim 24, wherein the one or more programming instructions stored thereon, when executed, further cause the processing device to:
provide a guidance to a user as one or more unexpected structures are encountered along the predetermined route.

27. The system of claim 26, wherein the one or more programming instructions stored thereon, when executed, further cause the processing device to:
modify the predetermined route to one or more predetermined back-up routes as the one or more unexpected structures are encountered along the predetermined route.

28. The system of claim 24, wherein the one or more programming instructions stored thereon, when executed, further cause the processing device to:
provide a guidance to a user as one or more unexpected conditions are encountered along the predetermined route.

29. The system of claim 28, wherein the one or more programming instructions stored thereon, when executed, further cause the processing device to:
modify the predetermined route to one or more predetermined back-up routes as the one or more unexpected conditions are encountered along the predetermined route.

30. The system of claim 29, wherein the one or more programming instructions stored thereon, when executed, further cause the processing device to:
override the guidance and continuing to direct the endoscope hardware to the one or more ROIs along the predetermined route.
